(19) United States
(12) Patent Application Publication
(10) Pub. No.: US 2008/0253960 A1
(43) Pub. Date: Oct. 16, 2008

(54) Lipoprotein-Based Nanoplatforms

(75) Inventors: Gang Zheng, West Grove, PA (US); Jerry D. Glickson, Ambler, PA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: The Trustees of the University of Pennsylvania Center for Technology Transfer, Philadelphia, PA (US)

(21) Appl. No.: 11/547,015

(22) PCT Filed: Apr. 1, 2005
(86) PCT No.: PCT/US05/11289

§ 371 Date: Mar. 17, 2008

Related U.S. Application Data
(60) Provisional application No. 60/558,502, filed on Apr. 1, 2004.
(60) Provisional application No. 60/643,825, filed on Jan. 14, 2005.

(51) Int. Cl. A61K 51/00; A61K 47/42; A61K 49/06; A61K 49/22; A61K 31/337; A61K 31/704; A61K 31/164; A61K 31/433; A61K 31/137; A61K 31/138; A61K 31/675; A61K 49/00; A61K 49/04; A61K 38/02

(52) U.S. Cl. 424/1.11; 514/773; 514/2; 424/9.3; 424/9.4; 424/9.5; 424/9.1; 424/9.36; 514/449; 514/90; 514/34; 514/651; 514/620; 514/653; 514/363

(57) ABSTRACT

The present invention provides a non-naturally occurring lipoprotein nanoplatform (“LBNP”) comprising at least one cell surface receptor ligand; at least one lipoprotein; and at least one diagnostic agent and/or at least one therapeutic agent. In embodiments of the present invention, the cell surface receptor ligand is not a low-density lipoprotein receptor ligand and the cell surface receptor ligand is covalently bonded to the apoprotein. The present invention also provides pharmaceutical formulations comprising LBNPs and methods of making the LBNPs.

### BACKGROUND OF THE INVENTION

1. Field of the Invention

The invention relates to non-naturally occurring lipoprotein nanoplatforms (“LBNP”) that allow targeted delivery of active agents. The active agents can be located in the core or the surface of the nanoplatform, whereas cell surface receptor ligands are attached to the apoprotein surface of the nanoplatform.

2. Background Art

### Nanoplatform

Nanoplatforms are nanoscale structures that are designed as general platforms to create a diverse set of multifunctional diagnostic and therapeutic devices. Such nanoscale devices typically have dimensions smaller than 100 nm and thus are comparable in size to other biological entities. They are smaller than human cells (10,000 to 20,000 nm in diameter) and organelles and similar in size to large biological macromolecules such as enzymes and receptors. Hemoglobin, for example, is approximately 5 nm in diameter, while the lipid bilayer surrounding cells is on the order of 6 nm thick. Nanoscale devices smaller than 50 nm can easily enter most cells, while those smaller than 20 nm can transit out of blood vessels (NIH/NCI Cancer Nanotechnology. NIH Publication No 04-5489 (2004)). As a result, nanodevices can readily interact with biomolecules both on the cell surface and within the cell, often in ways that do not alter the behavior and biochemical properties of those molecules. Thus, nanodevices offer an entirely unique vantage point from which to view and manipulate fundamental biological pathways and processes. Most of the multifunctional nanoplatforms reported so far are made of synthetic nanostructure s₁ such as dendrimers (spherical, branched polymers) (Quintana, A. et al. Journal of the American Chemical Society. 2003 125(26):7860-5), polymeric (Xu, H., Aylott, J. W. & Kopelman, R. Analyst. 2002 November; 127(11):1471-7, Pan, D., Turner, J. L. & Wooley, K. L. Chemical Communications, 2400-2401 (2003)) and ceramic (Kasili, P. M., Journal of the American Chemical Society. 2004 126(9):2799-806, Ruoslahti, E. Cancer Cell. 2002; 2(2):97-8) nanoparticles, perfluorocarbon emulsions (Anderson, S. A. et al., Magnetic Resonance in Medicine. 2000 September; 44(3):433-9) and cross-linked liposomes (Hood, J. D. et al., Science. 2002; 296(5577):2404-7, Li, L. et al., International Journal of Radiation Oncology, Biology, Physics. 2004 15; 58(4):1215-27).

One common concern among these synthetic nanoplatform designs is a biocompatibility issue, which is closely associated with short-term and long-term toxicity. Natural nanostructures could offer a solution to this biocompatibility problem. However, nanoplatforms made of naturally occurring nanostructures are rare.

The basic requirements for an ideal nanoplatform are as follows: (1) Uniform size distribution (<100 nm); 2) Large payload; 3) Multivalency for high binding affinity; 4) Platform technologies via modularity and multifunctionality; 5) Stable and long circulating time; and 6) Biocompatible, biodegradable and nontoxic. Although some of the existing synthetic nanoplatforms meet some of these criteria, one common concern among these nanoplatform designs is the biocompatibility issue, which is closely associated with short-term and long-term toxicity. Natural nanostructures could offer a solution to the biocompatibility problem. However, nanoplatforms made of naturally occurring nanostructures are rare. In one example, empty RNA virus capsules from cowpea mosaic virus and flockhouse virus served as potential nanodevices (Raja K. S. et al., Biomacromolecules 2003; 4(3):472-6). The premise is that 60 copies of coat protein that assemble into a functional virus capsule offer a wide range of chemical functionality that could be used to attach homing molecules—such as monoclonal antibodies or cancer cell-specific receptor antagonist and reporter molecules—such as magnetic resonance imaging (MRI) contrast agents to the capsule surface, and to load therapeutic agents inside the capsule. Unfortunately, even though the human body has quite a tolerance for viruses of plant origin; the immunologic effect associated with these nanodevices is undesirable.

### Targeted Delivery

Although the effect of a particular pathology often is manifest throughout the body of the afflicted person, generally, the underlying pathology may affect only a single organ or tissue. It is rare, however, that a drug or other treatment will target only the diseased organ or tissue. More commonly, treatment results in undesirable side effects due, for example, to generalized toxic effects throughout the patient's body. It would be desirable to selectively target organs or tissues, for example, for treatment of diseases associated with the target organ or tissue. It is also desirable to selectively target cancerous tissue in the body versus normal tissue.

Most therapeutic substances are delivered to the target organ or tissue through the circulation. The endothelium, which lines the internal surfaces of blood vessels, is the first cell type encountered by a circulating therapeutic substance in the target organ or tissue. These cells provide a target for selectively directing therapies to an organ or tissue.

Endothelium can have distinct morphologies and biochemical markers in different tissues. The blood vessels of the lymphatic system, for example, express various adhesion proteins that serve to guide lymphocyte homing. For example, endothelial cells present in lymph nodes express a cell surface marker that is a ligand for L-selectin and endothelial cells in Peyer's patch venules express a ligand for the α₄β₇ integrin. These ligands are involved in specific lymphocyte homing to their respective lymphoid organs. Thus, linking a drug to L-selectin or to the α₄β₇ integrin may provide a means for targeting the drug to diseased lymph nodes or Peyer's patches, respectively, provided that these molecules do not bind to similar ligands present in a significant number of other organs or tissues. Certain observations of lymphocyte circulation suggest that organ and tissue specific endothelial markers exist. Similarly, the homing or metastasis of particular types of tumor cells to specific organs or tissues further suggests that organ and tissue specific markers exist.

Targeted delivery to specific tissues, including cancerous tissue, is needed to eliminate undesirable side effects associated with unspecific delivery.

Recognizing the urgent need for delivery vehicles that specifically target desired tissues, the inventors of the present application have developed a unique lipoprotein based nanoplatform whose versatility makes it useful for a variety of applications.

### SUMMARY OF THE INVENTION

The invention relates to a non-naturally occurring lipoprotein nanoplatform comprising (a) at least one lipid; (b) at least one active cell surface receptor ligand; (c) at least one apoprotein; and (c) at least one active agent, wherein the active cell surface receptor ligand is not a low-density lipoprotein receptor ligand or a high-density lipoprotein receptor ligand and wherein the active cell surface receptor ligand is covalently bound to said apoprotein, and wherein the components (a), (b), (c) and (d) associate to form a non-naturally occurring lipoprotein nanoplatform.

### BRIEF DESCRIPTION OF THE DRAWINGS

FIG. 1 Schematic of an embodiment of a lipoprotein based nanoplatform of the present invention.

FIG. 2 Folate receptor pathway.

FIG. 3 Structures of pyropheophorbide cholesterol ester left) and Pc4 (right).

FIG. 4 Porphyrin molecules.

FIG. 5 Chemical transformations of chlorophyll a.

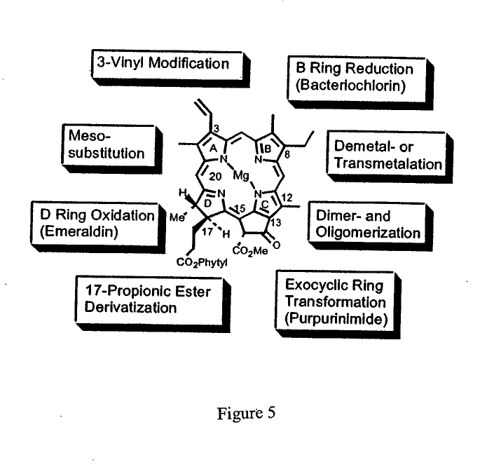

FIG. 6 Confocal images of HepG2 tumor cells incubated with unlabeled LDL as control (B), r-(Pyro-CE)-LDL (D), r-(Pyro-CE)-LDL with unlabeled LDL as inhibitors (F), non-LDL-reconstituted Pyro-CE for comparison (H), as well as the corresponding bright field images (A, C, E, G).

FIG. 7 Fluorescent images of r-Pyro-CE-LDL.

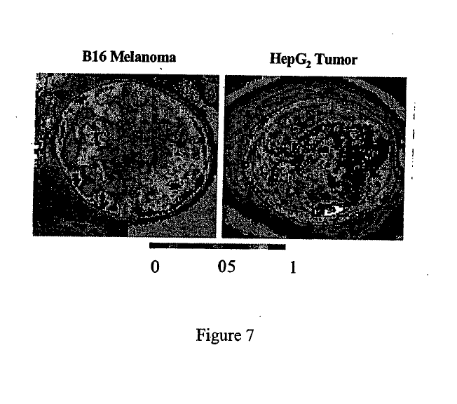

FIG. 8 Quantitative analysis of LDLR in B16 and HepG2 cells.

FIG. 9 Synthesis of (tBu)₄SiPc-BOA.

FIG. 10 Absorption and fluorescence spectra of (tBu)₄SiPcBOA.

FIG. 11 Confocal fluorescence images of HepG2 cells incubated w/wt fluorescent probes (B, D, F, H, J) as well as the corresponding bright field images (A, C, E, G, I). A, B, cell alone control; C, D, cell+85 μg/mL r-SiPcBOA-LDL; E, F, cell+85 μg/mL probe+50-fold over excess native LDL; G, H, cell+170 μg/mL r-SiPcBOA-LDL-AcLDL; I, J, cell+432 μg/mL (tBu)₄SiPcBOA (same amount of (tBu)₄SiPcBOA as in r-SiPcBOA-LDL.

FIG. 12 EM images of native LDL (left) and r-SiPc-BOA-LDL (right).

FIG. 13 In vitro PDT response of HepG2 cells to r-SiPcBOA-LDL. Average colony numbers+SEMs are shown. *, Significance at p<0.0125.

FIG. 14 MALDI-TOF mass spectrum of DTPA-Bis(stearylamide). Expanded insert displays dominant molecular ion peak at 894.5.

FIG. 15 T1-weighted axial spin-echo images through the abdomen (A,C,E) and lower flank (B,D,F) of nude mice with subcutaneous implanted Hep-G2 Tumor. Images A and B are from a control mouse while images C,D and E,F are from a mouse 5 and 24 hours, respectively, following the intravenous administration of Gd-DTPA-bis(stearylamide)LDL. (Arrow indicates tumor; arrow head indicates liver parenchyma).

FIG. 16 Schematic diagram of apoB-100 structure.

FIG. 17 Two isoforms of folate conjugates.

FIG. 18 Indirect spectrophotometric determination of DTPA ligand.

### DETAILED DESCRIPTION OF THE PRESENT INVENTION

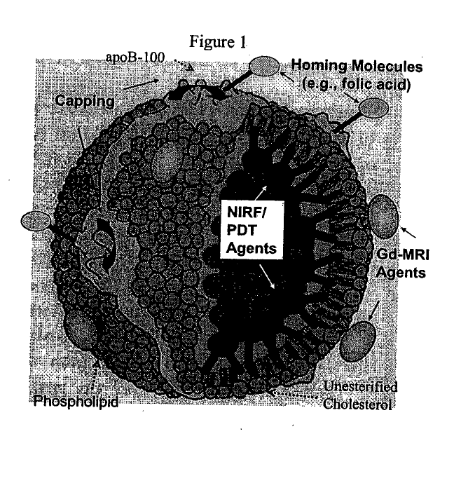

FIG. 1 demonstrates an embodiment of the present invention. Specifically, FIG. 1 illustrates a low-density lipoprotein-based nanoplatform (“LBNP”) that can be used to create a diverse set of multifunctional cancer diagnostic and therapeutic devices. The low-density lipoprotein (LDL) particle is a naturally occurring nanostructure typically with a diameter of ˜22 nm. It contains a lipid core of some 1500 esterified cholesterol molecules and triglycerides. A shell of phospholipids and unesterified cholesterol surrounds this highly hydrophobic core. The shell also contains a single copy of apoB-100, which is recognized by the LDL receptor (LDLR). Diverse targeting is achieved by conjugating certain tumor-homing molecules (e.g., folic acid) to the Lys residues exposed on the apoB-100 surface optionally followed by capping the remaining unreacted Lys residues. LDLR binding is turned off and the modified LDL particles are redirected to the desired cancer signatures and/or specific tissues, i.e., molecules that are selectively overexpressed in various types of cancer cells. In particular embodiments, the multifunctionality of LBNP provides targeted delivery of active agents including, but not limited to, diagnostic and/or therapeutic agents.

Such diagnostic agents include, but are not limited to, magnetic resonance imaging (MRI) agents, near-infrared fluorescence (NIRF) probes and photodynamic therapy (PDT) agents.

In particular embodiments, the cholesterol esters inside the LDL lipid core are replaced with lipophilic agents. In additional embodiments, active agents are attached to the surface of the LBNPs of the present invention.

Thus, accumulation of the MRI/NIRF probes in target cells provides a facile mechanism for amplification of the MRI/NIRF detectable signal and affords opportunities to combine the strength of both MRI (high resolution/anatomic) and NIRF (high sensitivity), whereas the selective delivery of PDT agents to tumors via these pathways also provides a facile transition between the detection and treatment. Finally, being endogenous carriers, LDL particles are not immunogenic and escape recognition by the reticuloendothelial system, thus the LBNP platform provides a solution to common problems associated with most synthetic nanodevices, namely biocompatibility and toxicity.

In order to develop a nanoplatform that can be used to target various types of cancers other than those overexpressing LDL receptors, the LDL receptor binding sites on the LDL particles are blocked and these nanoparticles are retargeted to alternate cell surface receptors. There are at least three ways to block LDL receptor binding. One involves modifying the protein residues at the receptor binding sites by reductive methylation (Lund-Katz S. et al., Journal of Biological Chemistry 1988; 263(27):13831-8); a second method is to impede receptor binding by changing the lipid core constituents (Aviram M., Journal of Biological Chemistry 1988; 263(32):16842-8). A third is by attaching at least one tissue/tumor homing molecule to the apoB-100 protein amino acid residues. One or more of these techniques can be practiced according to the methods of the present invention.

The present invention therefore provides a non-naturally occurring lipoprotein nanoplatform comprising at least one lipid, at least one active cell surface receptor ligand, at least one apoprotein; and at least one active agent; wherein the active cell surface receptor ligand is not a low-density lipoprotein receptor ligand or a high-density lipoprotein receptor ligand and wherein the active cell surface receptor ligand is covalently bound to said apoprotein, and wherein the components form a non-naturally occurring lipoprotein nanoplatform.

The invention provides LBNPs comprising lipids such as phosphatidylcholine, lysophosphatidylcholine, phosphatidyl-ethanolamine, phosphatidylserine, phosphatidylinositol, as well as combinations thereof.

The naturally occurring lipoprotein particles each have characteristic apoproteins, and percentages of protein, triacylglycerol, phospholipids and cholesterol. VLDL particles can contain about 10% protein, about 60% triacylglycerols, about 18% phopholipids and about 15% cholesterol. LDL particles can contain about 25% protein, about 10% triacylglycerols, about 22% phopholipids and about 45% cholesterol. HDL particles can contain about 50% protein, about 3% triacylglycerols, about 30% phopholipids and about 18% cholesterol. Likewise, the LBNPs of the invention contain different percentages of the above constituents, and may not even contain any percentage of triacylglycerol or cholesterol.

In certain embodiments, the LBNPs of the present invention are from 5 to 100 nm in diameter, and preferably from 8 to 80 nm in diameter.

In particular embodiments, the LBNPs of the present invention contain cell surface receptor ligands over-expressed in cancer cells. In embodiments, the LBNPs of the present invention contain cell surface receptor ligands that are ligands for a receptor over-expressed in cardiovascular plaques.

In additional embodiments, the LBNPs of the present invention contain cell surface receptor ligands that target a specific tissue.

In certain embodiments, the LBNPs of the present invention contain lipophilic compounds in the core of the LBNP.

In certain embodiments, the LBNPs of the present invention contain active agents that are molecules comprising a lipophilic and a hydrophilic component that are located on the surface of the LBNP.

In particular embodiments, the LBNPs of the present invention contain an active agent that is a diagnostic agent or a therapeutic agent. Preferably, the diagnostic agent is a contrast agent, a radioactive label and/or a fluorescent label.

In certain embodiments, the LBNPs of the present invention contain anticancer agents. Such active agents can be a chemotherapeutic agent, a photodynamic therapy agent, a boron neutron capture therapy agent or a radionuclide for radiation therapy. In embodiments, the anticancer agent is selected from the group consisting of alkylators, anthracyclines, antibiotics, aromatase inhibitors, bisphosphonates, cyclo-oxygnase inhibitors, estrogen receptor modulators, folate antagonists, inorganic arsenates, microtubule inhibitors, modifiers, nitrosureas, nucleoside analogs, osteoclast inhibitors, platinum containing compounds, retinoids, topoisomerase 1 inhibitors, tyrosine kinase inhibitors, and epidermal growth factor inhibitors.

In additional embodiments, the LBNPs of the present invention contain a therapeutic agent selected from the group consisting of an antiglaucoma drug, an anti-clotting agent, an anti-inflammatory drug, an anti-asthmatic, an antibiotic, an antifungal or an antiviral drug.

The present invention also provides LBNPs that contain an apoprotein, wherein the apoprotein is selected from the group consisting of apoB-100, apoB-48, apoC, apoE and apoA.

The invention also provides pharmaceutical formulations comprising the LBNPs of the present invention.

The invention further provides methods of making the LBNPs of the present invention comprising reconstituting a lipoprotein particle with an active agent and attaching a cell surface receptor ligand to the apoprotein of the reconstituted lipoprotein particle. In embodiments, the lipoprotein particle is an LDL particle or an HDL particle.

### Lipoprotein Particles

Lipoprotein particles are a class of naturally occurring nanostructures. Cholesterol and triacylglycerols are transported in body fluids in the form of lipoprotein particles. Each particle consists of a core of hydrophobic lipids surrounded by a shell of more polar lipids and proteins. The protein components of these macromolecular aggregates have two roles: they solubilize hydrophobic lipids and contain cell-targeting signals. Lipoprotein particles are classified according to increasing density: chylomicrons, chylomicron remnants, very low density lipoprotein (VLDL), intermediate-density lipoproteins (IDL), low-density lipoprotein (LDL), and high density lipoproteins (HDL) (See Table 1). Accordingly, each of them is different in size, and most of them have nanostructures (<100 nm) with the exception of chylomicrons and chylomicron remnants.

TABLE 1
Size and class of lipoproteins

| Lipoproteins | Major Core Lipids | Apoproteins | Size | |
|---|---|---|---|---|
| Chylomicrons | Dietary | B-48, C, E | 75-1200 | nm |
| | triacylglycerols, | | | |
| | cholesterol esters | | | |
| VLDL | Endogenous | B-100, C, E | 30-80 | nm |
| | triacylglycerols | | | |
| IDL | Endogenous | B-100, E | 25-35 | nm |
| | cholesterol esters | | | |
| LDL | Endogenous | B-100 | 18-25 | nm |
| | cholesterol esters | | | |
| HDL | Endogenous | A, C, E | 8-12 | nm |
| | cholesterol esters | | | |

### Low Density Lipoprotein (LDL) Particles

LDL is the principal carrier of cholesterol in human plasma and delivers exogenous cholesterol to cells by endocytosis via the LDLR. The LDL particle is a naturally occurring nanostructure typically with a diameter of ˜22 nm. It contains a lipid core of some 1500 esterified cholesterol molecules and triglycerides. A shell of phospholipids and unesterified cholesterol surrounds this highly hydrophobic core. The shell also contains a single copy of apoB-100, which is recognized by the LDLR.

### High Density Lipoprotein (HDL) Particles

Plasma HDL is a small, spherical, dense lipid-protein complex that is approximately half lipid and half protein. The lipid component consists of phospholipids, free cholesterol, cholesteryl esters, and triglycerides. The protein component includes apo A-I (molecular weight, 28,000 Daltons) and apo A-II (molecular weight, 17,000 Daltons). Other minor but important proteins are apo E and apo C, including apo C-I, apo C-II, and apo C-III.

HDL particles are heterogeneous. They can be classified as a larger, less dense HDL2 or a smaller, more dense HDL3. Normally, most of the plasma HDL is found in HDL3. HDL is composed of 4 apolipoproteins per particle. HDL may be composed of both apo A-I and apo A-II or of apo A-I only. HDL2 is predominantly apo A-I only, and HDL3 is made of both apo A-I and apo A-II. HDL particles that are less dense than HDL2 are rich in apo E.

### Non-Naturally Occurring LBNPs

Accordingly, the present invention provides a series of nanoplatforms with different sizes that can be made from all the lipoproteins (listed in Table 1). Since each of their apoproteins is targeted to specific receptors, if these are blocked, the lipoproteins can be retargeted to alternate receptors. Moreover, in certain embodiments, both the lipoprotein hydrophobic core and phospholipids monolayer can be modified to carry large payloads of diagnostic and/or therapeutic agents making them exceptional multifunctional nanoplatforms. In certain embodiments, the LBNPs of the present invention contain one or more homing molecules. The LBNPs also can carry payloads of one or more active agents. The LBNPs can also contain a cell death sensor so such LBNP can simultaneously perform diagnosis, treatment as well as therapeutic response monitoring functions.

The invention provides non-naturally occurring nano-platforms that are based on naturally occurring lipoprotein particles, described above. The term “non-naturally occurring” refers to nanoplatforms that do not exist innately in the human body. Such non-naturally occurring LBNPs can contain one or more components of naturally occurring lipoprotein particles. For example, some or all of the cholesterol esters that exist in the core of naturally occurring LDL and HDL particles are replaced with active agents, but lipids comprising the outer surface of the particle are not replaced. Likewise, the core of the naturally occurring lipoprotein particles can remain intact, but an active agent is attached to the surface of the lipoprotein particle. Additionally, in certain embodiments of the present invention, the naturally occurring cell surface receptors of the lipoprotein particle (eg. LDL and HDL) cell surface receptor ligands to the surface of the apoprotein of the naturally occurring lipoprotein particle.

The non-naturally occurring LBNPs of the present invention are preferably from 5 nm to 500 nm, in diameter, from 5 nm to 100 nm in diameter; and from 5 nm to 80 nm in diameter.

There are several distinct advantages for using lipoprotein based particles for targeted delivery. One advantage of the lipoprotein-based nanoplatforms (LBNP) of the present invention is that they are completely compatible with the host immune system, and they are also completely biodegradable. They also provide a recycling system for accumulation of large quantities of diagnostic or therapeutic agents in the target cells. Specifically, being endogenous carriers, lipoprotein particles are not immunogenic and escape recognition by the reticuloendothelial system (RES).

Other advantages include: 1) lipoproteins, which are a physiological carrier, are not cleared by the reticuloendothelial system (RES) and may prolong the serum half-life of drugs/probes by incorporation into it; 2) drug/probe sequestration in the lipid core space provides protection from serum enzyme and water; 3) the availability of the array of lipoproteins provide a series of nanoplatforms with size ranging from 5 nm to 500 nm.

Each lipoprotein particle in Table 1 contains at least one apoprotein that aids in targeting cell surface receptors. For example, LDL contains apoB-100. The mature apoB-100 molecule comprises a single polypeptide chain of 4536 amino acid residues. Chemical modification of functional groups in the apoB-100 molecule has shown that the electrostatic interaction of domains containing basic Lys and Arg residues with acidic domains on the LDLR is important to the binding process. (Mahley, R. W. et al., Journal of Biological Chemistry. 1977; 252(20):7279-87). The involvement of Lys in the LDLR binding process is particularly important. There are two types of Lys residues on the apoB-100 protein; “active” Lys have a pK of about 8.9, while “normal” Lys have a pK of about 10.5. (Lund-Katz, S. et al., Journal of Biological Chemistry. 1988; 263(27):13831-8). ApoB-100 contains 53 active and 172 normal Lys residues are exposed on the surface of LDL with the remaining 132 Lys residues (a third of total Lys) which are present in apoB-100 being buried and unavailable for reaction. Effective Lys modifications include reaction of LDL with organic acid anhydrides (acetylation or maleylation) and reaction with aldehydes, such as malondialdehyde. (Brown, M. S. et al., Journal of Supramolecular Structure. 1980; 13(1):67-81). Reductive methylation with formaldehyde and sodium cyanoboronhydride is also an effective Lys capping technique. (Lund-Katz, S et al., Journal of Biological Chemistry. 1988; 263(27):13831-8.) Almost all Lys residues exposed on the LDL surface (two third of total Lys: 225) can be capped by these procedures. The fact that Lys residues can be transformed without significant alteration in their pK means that such LDL modification does not alter the conformation of the apoB-100. However, these modifications do impair the ability of apoB-100 on LDL to bind to the LDLR. Lund-Katz et al., demonstrated that when about 20% of the Lys are capped, binding to the LDLR is essentially abolished (Lund-Katz, S. et al. Journal of Biological Chemistry. 1988 Sep. 25; 263(27):13831-8.). The ability of LDL to bind to the LDLR appears to be reduced by 50% when about 8% of the Lys residues are methylated.

Moreover, the chemical modification of apoB-100 described above often directs LDL particles to non-lipoprotein receptors. For example, acetylation of LDL induces rapid uptake by scavenger receptors on endothelial liver cells (Pitas, R. E. Journal of Cell Biology. 1985 January; 100(1):103-17). Lactosylation of LDL induces rapid, galactose-specific uptake by Kupffer and parenchymal liver cells, respectively (Bijsterbosch, M. K. et al., Advanced Drug Delivery Reviews 5, 231-251 (1990).

The apoproteins of all the lipoprotein particles listed in Table 1 can also be modified to turn off targeting to its receptor, enabling redirection by an alternate cell surface receptor.

While the LBNPs of the present invention can be constructed using components isolated from naturally occurring LDLs, HDLs, etc., the present invention also provides recombinant lipoproteins engineered to offer desirable surface modifications. Such recombinant lipoproteins can make these nanoparticles more consistent than the lipoproteins isolated from the human blood.

The starting materials of the non-naturally occurring LBNPs also contain at least one lipid that is on, for example, the outer layer of the particle. Lipids useful in the LBNPs of the present invention include, but are not limited to, amphipathic lipids. Phopholipids useful in the present invention include, but are not limited to, phosphatidylcholine, lysophosphatidylcholine, phosphatidylethanolamine, phosphatidylserine, phosphatidylinositol and combinations thereof.

The naturally occurring lipoprotein particles each have characteristic apoproteins, and percentages of protein, triacylglycerol, phospholipids and cholesterol. VLDL particles can contain about 10% protein, about 60% triacylglycerols, about 18% phopholipids and about 15% cholesterol. LDL particles can contain about 25% protein, about 10% triacylglycerols, about 22% phopholipids and about 45% cholesterol. HDL particles can contain about 50% protein, about 3% triacylglycerols, about 30% phopholipids and about 18% cholesterol. Likewise, the LBNPs also contain different percentages of lipids, and may even not contain any percentage of triacylglycerol or cholesterol.

### Homing Molecules

As used herein, the term “home” or “selectively home” means that a particular molecule binds relatively specifically to molecules present in specific organs or tissues following administration to a subject. In general, selective homing is characterized, in part, by detecting at least a two-fold greater selective binding of the molecule to an organ or tissue as compared to a control organ or tissue. In certain embodiments the selective binding is at least three-fold or four-fold greater as compared to a control organ or tissue.

In the case of tumor homing molecules, such molecules bind to receptors that are selectively over-expressed in particular cancer tissues. By over expression is meant at least one and one half greater expression in tumor tissue compared to normal tissue. In embodiments, expression is at least five times greater in tumor as compared to non-tumor.

In embodiments of the present invention, a homing molecule is attached to the lipoprotein of the LBNP of the present invention that targets specific tissues and tumors. A “homing molecue” refers to any material or substance that may promote targeting of tissues and/or receptors in vitro or in vivo with the compositions of the present invention. The targeting moiety may be synthetic, semi-synthetic, or naturally-occurring. The targeting moiety may be a protein, peptide, oligonucleotide, or other organic molecule. The targeting moiety may be an antibody (this term including antibody fragments and single chain antibodies that retain a binding region or hypervariable region). Materials or substances which may serve as targeting moieties include, but are not limited to, those substances listed in Table 2:

TABLE 2

| Targeting Moiety | Target(s) |
|---|---|
| Antibodies (and fragments such as Fab, | RES system |
| F(ab)′2, Fv, Fc, etc.) | |
| Epidermal growth factor (EGF) | Cellular receptors |
| Collagen | Cellular receptors |
| Gelatin | Cellular receptors |
| Fibrin-binding protein | Fibrin |
| Plasminogen activator | Thrombus |
| Urokinase inhibitor | Invasive cells |
| Somatostatin analogs | Cellular receptors |
| Lectin (WGA) | Axons |
| f-Met-Leu-Phe | Neutrophils |
| Selectin active fragments | Glycosyl structures |
| ELAM, GMP 140 | Leucocyte receptors |
| “RGD” proteins | Integrins, Granulocytes |
| IL-2 | Activated T-cell |
| CD4 | HIV infected cells |
| Cationized albumin | Fibroblasts |
| Carnitine | |
| Acetyl-, maleyl-proteins | Macrophage scavenger receptor |
| Hyaluronic acid | Cellular receptors |
| Lactosylceramide | Hepatocytes |
| Asialofoetuin | Hepatocytes |
| Arabinogalactan | Hepatocytes |
| Galactosylated particles | Kupffer cells |
| Terminal fucose | Kupffer cells |
| Mannose | Kupffer cells, macrophages |
| Lactose | Hepatocytes |
| Dimuramyl-tripeptide | Kupffer cells, macrophages |
| Fucoidin-dextran sulfate | Kupffer cells, macrophages |
| Sulfatides | Brain |
| Glycosyl-steroids | |
| Glycosphyngolipids | Other glycosylated structures |
| Hypoxia mediators | Infarcted tissues |
| Amphetamines | Nervous system |
| Barbiturates | Nervous system |
| Sulfonamides | |
| Monoamine oxidase inhibitor | Brain |
| substrates | |
| Chemotactic peptides | Inflammation sites |
| Muscarine and dopamine receptor | Nervous system |
| substrates | |

### Tumor Homing Molecules

Tumor homing molecules selectively bind to tumor tissue versus normal tissue of the same type. Such molecules in general are ligands for cell surface receptors that are over-expressed in tumor tissue. Cell surface receptors over-expressed in cancer tissue versus normal tissue include, but are not limited to, epidermal growth factor receptor (EGFR) overexpressed in anaplastic thyroid cancer and breast and lung tumors, metastin receptor overexpressed in papillary thyroid cancer, ErbB family receptor tyrosine kinases overexpressed in a significant subset of breast cancers, human epidemal growth factor receptor-2 (Her2/neu) overexpressed in breast cancers, tyrosine kinase receptor (c-Kit) overexpressed in sarcomatoid renal carcinomas, HGF receptor c-Met overexpressed in esophageal adenocarcinoma, CXCR4 and CCR7 overexpressed in breast cancer, endothelin-A receptor overexpressed in prostate cancer, peroxisome proliferator activated receptor delta (PPAR-delta) overexpressed in most colorectal cancer tumors, PDGFR A overexpressed in ovarian carcinomas, BAG-1 overexpressed in various lung cancers, soluble type II TGF beta receptor overexpressed in pancreatic cancer folate and integrin (e.g. αvβ₃)

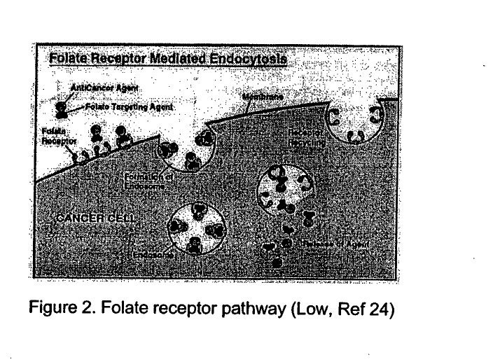

The folate receptor is a glycosylphosphatidylinositol-anchored glycoprotein with high affinity for the vitamin folic acid (Kd˜10⁻⁹ M) (Leamon, C. P. et al., Biochemical Journal. 1993 May 1; 291 (Pt. 3):855-60). Folate receptor has been identified as a tumor-marker, which is expressed at elevated levels relative to normal tissues on epithelial malignancies, such as ovarian, colorectal, and breast cancer (Wang, S. et al., Journal of Controlled Release. 1998 Apr. 30; 53(1-3):39-48). It has been shown that when folate is covalently linked to either a single molecule or assembly of molecules via its g-carboxyl moiety, its affinity for the cell surface receptors remains essentially unaltered. As shown in FIG. 2, following endocytosis and vesicular trafficking, much of the material is released into the cell cytoplasm. The unliated folate receptor may then recycle to the cell surface; thus, each folate receptor may bring many folate conjugates into the cell.

### Organ or Tissue Homing Molecules

The present invention provides molecules that selectively home to various organs or tissues, including to lung, skin, pancreas, retina, prostate, ovary, lymph node, adrenal gland, liver, breast, digestive system or renal tissue. For example, the invention provides using lung homing peptides such as those containing a GFE motif, including the peptides CGFECVRQCPERC and CGFELETC; skin homing peptides such as CVALCREACGEGC; pancreas homing peptides such as the peptide SWCEPGWCR; and retina homing peptides such as those containing an RDV motif, including the peptides CSCFRDVCC and CRDVVSVIC.

The invention also provides methods of using an organ homing molecule of the invention to diagnose or treat a pathology of the lung, skin, pancreas, retina, prostate, ovary, lymph node, adrenal gland, liver or gut by administering a molecule that homes to the selected organ or tissue to a subject having or suspected of having a pathology. For example, a pathology of lung, skin, pancreas, retina, prostate, ovary, lymph node, adrenal gland, liver or gut can be treated by administering to a subject having the pathology a LBNP comprising an appropriate organ homing molecule linked to a therapeutic agent. Similarly, a method of identifying a selected organ or tissue or diagnosing a pathology in a selected organ by administering to a subject a LBNP comprising an appropriate organ homing molecule linked to a detectable agent.

The LBNPs of the present invention can be used with organ and tissue homing molecules to target a moiety to a selected organ or tissue. The homing molecules employed in the invention include peptides that home to various normal organs or tissues, including lung, skin, pancreas, retina, prostate, ovary, lymph node, adrenal gland, liver or gut, and to organs bearing tumors, including to lung bearing lung tumors and to pancreas bearing a pancreatic tumor. For example, the invention includes the use of lung homing peptides, including the peptides CGFECVRQCPERC and CGFELETC, each of which contains a tripeptide GFE motif, and the peptide GIGEVEVC. The invention also includes the use of skin homing peptides such as the peptide CVALCREACGEGC; pancreas homing peptides such as the peptide SWCEPGWCR and retina homing peptides such as the peptides CSCFRDVCC and CRDVVSVIC, each of which contains a tripeptide RDV motif. Examples of peptides that home to prostate, ovary, lymph node, adrenal gland, liver and gut are also provided.

For convenience, the term “peptide” is used broadly herein to mean peptides, polypeptides, proteins and fragments of proteins and includes, for example, single-chain peptides. Other molecules useful in the invention include peptoids, peptidomimetics and the like. With respect to the organ or tissue homing peptides of the invention, peptidomimetics, which include chemically modified peptides, peptide-like molecules containing nonnaturally occurring amino acids, peptoids and the like, have the binding activity of an organ homing peptide upon which the peptidomimetic is derived (see, for example, “Burger's Medicinal Chemistry and Drug Discovery” 5th ed., vols. 1 to 3 (ed. M. E. Wolff; Wiley Interscience 1995), which is incorporated herein by reference). Peptidomimetics provide various advantages over a peptide, including that a peptidomimetic can be stable when administered to a subject, for example, during passage through the digestive tract and, therefore, useful for oral administration.

Methods for identifying a peptidomimetic are well known in the art and include, for example, the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). This structural depository is continually updated as new crystal structures are determined and can be screened for compounds having suitable shapes, for example, the same shape as an organ or tissue homing molecule, as well as potential geometrical and chemical complementarity to a target molecule bound by an organ or tissue homing peptide. Where no crystal structure of a homing peptide or a target molecule, which binds an organ or tissue homing molecule, is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of an organ or tissue homing molecule.

Selective homing of a molecule to a selected organ or tissue can be due to selective recognition by the molecule of a particular cell target molecule such as a cell surface protein present on a cell in the organ or tissue. Selectivity of homing is dependent on the particular target molecule being expressed on only one or a few different cell types, such that the molecule homes to only one or a few organs or tissues. In this regard, most different cell types, particularly cell types that are unique to an organ or tissue, can express unique target molecules.

Examples of peptide motifs that have been identified as useful for homing to particular organs or tissue include those listed in Table 3.

TABLE 3
ORGAN/MOTIF

GUT
YSGKWGK
GISALVLS
SRRQPLS
MSPQLAT
MRRDEQR
QVRRVPE
VRRGSPQ
GGRGSWE
FRVRGSP
RVRGPER
LIVER
VKSVCRT
WRQNMPL
SRRFVGG
ALERRSL
ARRGWTL
PROSTATE
SMSIARL
VSFLEYR
RGRWLAL
ADRENAL GLAND
LMLPRAD
LPRYLLS
OVARY
EVRSRLS
VRARLMS
RVGLVAR
RVRLVNL
PANCREAS
SWCEPGWCR
SKIN
CVALCREACGEGC
CSSGCSKNCLEMC
CIGEVEVC
CKWSRLHSC
CWRGDRKIC
CERVVGSSC
CLAKENVVC
LUNG
CTLRDRNC
CGKRYRNC
CLRPYLNC
CGFELETC
CTVNEAYKTRMC
CRLRSYGTLSLC
CRPWHNQAHTEC
CGFECVRQCPERC

The invention includes the use of lung homing peptides such as CGFECVRQCPERC and CGFELETC, which share a GFE motif; CTLRDRNC; and CIGEVEVC which contains an EVE motif that is similar to the ELE motif present in CGFELETC.

Preferably, the invention also can use skin homing peptides such as CVALCREACGEGC. The invention further provides LBNP with pancreas homing peptides such as SWCEPGWCR. Retina homing peptides such as CSCFRDVCC and CRDVVSVIC can also be used in conjunction with the LBNP of the present invention. Prostate homing peptides such as SMSIARL and VSFLEYR, can also be used with the LBNP of the present invention. Also provided are ovary homing peptides such as RVGLVAR and EVRSRLS. The invention also can use adrenal gland homing peptides such as LMLPRAD and LPRYLLS, which share a LPR motif or the peptides R(Y/F)LLAGG and RYPLAGG, which share the motif LAGG. In addition, lymph node homing peptides, such as AGCSVTVCG can be used in conjunction with the present invention. The invention also can use gut homing peptides such as YSGKWGK and YSGKWGW.

### Cardiovascular Plaque Homing Molecules

Atherosclerosis plaques are known to over-express certain receptors, such as CX3CL1. The invention therefore includes ligands for receptors over-expressed on such plaques.

### Active Agents

Lipophilic Compounds

A variety of active agents can be delivered via the LBNPs of the present invention. In embodiments, the active agent is located in the core of the LBNP, which is generally lipophilic. Lipophilic compounds are therefore able to be delivered via the LBNPs of the present invention. The LBNPs of the present invention can be used with active agents that are inherently lipophilic or can be made lipophilic by chemical modification, discussed in more detail below.

The term “lipophilic compound” or “lipophilic drug” is defined as a compound or drug which in its non-ionized form is more soluble in lipid or fat than in water. Examples of lipophilic compounds include, but are not limited to, acetanilides, anilides, aminoquinolines, benzhydryl compounds, benzodiazepines, benzofurans, cannabinoids, cyclic peptides, dibenzazepines, digitalis gylcosides, ergot alkaloids, flavonoids, imidazoles, quinolines, macrolides, naphthalenes, opiates (or morphinans), oxazines, oxazoles, phenylalkylamines, piperidines, polycyclic aromatic hydrocarbons, pyrrolidines, pyrrolidinones, stilbenes, sulfonylureas, sulfones, triazoles, tropanes, and vinca alkaloids.

A variety of tests can be used to determine lipophilicity. A common test protocol is measurement of the octanol-water partition coefficient (P_{OW}, K_{OW}), which is a measure of lipophilicity by determination of the equilibrium distribution between octan-1-ol and water. Lipophilic drugs are those drugs that preferably partition into the octanol component.

Pharmaceutically active lipophilic drugs which may be incorporated into targeted drug delivery complexes of the invention include drugs for the treatment of cancer and glaucoma, immunoactive agents, antineoplastic agents, anticholinergic and cholinomimetic agents, antimuscarinic and muscarinic agents, antiadrenergic and antiarrhythmics, antihypertensive agents, anti-inflammatory drugs, antibiotic drugs, anti-fungal drugs, steroids, anti-histamines, anti-asthmatics, sedatives, anti-epileptics, anesthetics, hypnotics, antipsychotic agents, neuroleptic agents, antidepressants, anxiolytics, anti-convulsant agents, neuron blocking agents, narcotic antagonists, analgesics, anti-proliferative agents, anti-viral drugs, hormones, and nutrients.

Examples of anti-cancer drugs include but are not limited to paclitaxel, docosahexaenoic acid (DHA)-paclitaxel conjugates, cyclophosphoramide, betulinic acid, and doxorubicin (see, e.g. U.S. Pat. No. 6,197,809 to Strelchenok).

Examples of anti-glaucoma drugs include but are not limited to β-blockers such as timolol-base, betaxolol, atenolol, livobunolol, epinephrine, dipivalyl, oxonolol, acetazolamide-base and methzolamide.

Examples of anti-inflammatory drugs include but are not limited to steroidal drugs such as cortisone and dexamethasone and non-steroidal anti-inflammatory drugs (NSAID) such as piroxicam, indomethacin, naproxen, phenylbutazone, ibuprofen and diclofenac acid. Examples of anti-asthmatics include but are not limited to prednisolone and prednisone. (See also U.S. Pat. No. 6,057,347).

An example of an antibiotic drug includes but is not limited to chloramphenicol. Examples of anti-fungal drugs include but are not limited to nystatin, amphotericin B, and miconazole. Examples of an anti-viral drug includes but is not limited to Acyclovir™ (Glaxo Wellcome, U.K.).

Examples of steroids include but are not limited to testosterone, estrogen, and progesterone. Examples of anti-allergic drugs include but are not limited to pheniramide derivatives. Examples of sedatives include but is not limited to diazepam and propofol.

Compounds to be delivered that are not ordinarily lipophilic may be used in the present invention by fusing or covalently coupling them to one or more lipophilic molecule(s) to produce an amphiphathic compound. Such lipophilic molecule preferably contain at least one long hydrocarbon chain (>C₁₀) which is either bent by a cis-double bond or branched by at least one side chain, for example. Such molecules include, but are not limited to, cholesterol oleate, oleate, cholesterol laurate or phytol. Sterols and fatty acids can also be used.

Nucleic acids may be delivered as “lipophilic” compounds by complexing the nucleic acid with a cationic lipid to form a lipophilic complex which can then be incorporated into the neutral lipid core of the particle.

Hydrophilic Agents with Lipid Anchors

In addition to active agents that are lipophilic and can be loaded into the core the LBNPs of the present invention, the invention also includes active agents that can be loaded onto the surface of the apoproteins of the present invention. Such active agents can be hydrophilic with a lipid anchor. For example, the LBNPs of the present invention can be modified to include a lipophilic chelator, such lipophilic chelators are well known in the art. For example, the lipophilic chelator, DTPA Bis (stearylamine), can be incorporated into an LDL particle using standard techniques. Likewise 1,1-dioctadecyl-3,3,3,3-tetramethylindocarbocyanine perchlorate (DiI), be used as a lipid-anchored, carbocanine based optical probe known to intercolate into the LDL phospholipid monolayer and can be used in the LBNPs of the present invention.

Similarly, near infrared (“NIR”) probes such as tricarbocyanine dyes, which are NIR fluorophores, can be modified to include a lipid-chelating anchor that allows such probes to be anchored to the LBNPs of the present invention. Any such lipid-chelating anchors can be used, for example, a cholesteryl laurate moiety can be attached to the NIR probes to anchor them to the LBNPs of the present invention. (Zheng et al., Bioorg. & Med. Chem. Lett. 12:1485-1488 (2002).

### Diagnostic Agents

In one embodiment, an active agent can be a detectable agent such as a radionuclide or an imaging agent, which allows detection or visualization of the selected organ or tissue. Thus, the invention provides a LBNP comprising a lung, skin, pancreas, retina, prostate, ovary, lymph node, adrenal gland, liver or gut homing molecule. The type of detectable agent selected will depend upon the application. For example, for an in vivo diagnostic imaging study of the lung in a subject, a lung homing molecule can be linked to a LBNP comprising an agent that, upon administration to the subject, is detectable external to the subject. For detection of such internal organs or tissues, for example, the prostate, a gamma ray emitting radionuclide such as indium-113, indium-115 or technetium-99 can be conjugated with a LBNP that is linked to a prostate homing molecule and, following administration to a subject, can be visualized using a solid scintillation detector. Alternatively, for organs or tissues at or near the external surface of a subject, for example, retina, a fluorescein-labeled retina homing molecule can be used such that the endothelial structure of the retina can be visualized using an opthalamoscope and the appropriate optical system.

Molecules that selectively home to a pathological lesion in an organ or tissue similarly can be used in the LBNP of the invention to deliver an appropriate detectable agent such that the size and distribution of the lesion can be visualized. For example, where an organ or tissue homing molecule homes to a normal organ or tissue, but not to a pathological lesion in the organ or tissue, the presence of the pathological lesion can be detected by identifying an abnormal or atypical image of the organ or tissue, for example, the absence of the detectable agent in the region of the lesion.

A detectable agent also can be an agent that facilitates detection in vitro. For example, a LBNP conjugate comprising a homing molecule and an enzyme, which produces a visible signal when an appropriate substrate is present, can detect the presence of an organ or tissue to which the homing molecule is directed. Such a conjugate, which can comprise, for example, alkaline phosphatase or luciferase or the like, can be useful in a method such as immunohistochemistry. Such a conjugate also can be used to detect the presence of a target molecule, to which the organ homing molecule binds, in a sample, for example, during purification of the target molecule.

Additional diagnostic agent include contrast agents, radioactive labels and fluorescent labels. Preferred contrast agent are optical contrast agents, MRI contrast agents, ultrasound contrast agents, X-ray contrast agents and radio-nuclides.

### Therapeutic Agents

A therapeutic agent can be any biologically useful agent that exerts its function at the site of the selected organ or tissue. For example, a therapeutic agent can be a small organic molecule that, upon binding to a target cell due to the linked organ homing molecule, is internalized by the cell where it can effect its function. A therapeutic agent can be a nucleic acid molecule that encodes a protein involved in stimulating or inhibiting cell survival, cell proliferation or cell death, as desired, in the selected organ or tissue. For example, a nucleic acid molecule encoding a protein such as Bcl-2, which inhibits apoptosis, can be used to promote cell survival, whereas a nucleic acid molecule encoding a protein such as Bax, which stimulates apoptosis, can be used to promote cell death of a target cell.

A particularly useful therapeutic agent that stimulates cell death is ricin, which, when linked to an organ homing molecule of the invention, can be useful for treating a hyperproliferative disorder, for example, cancer. A LBNP comprising an organ homing molecule of the invention and an antibiotic, such as ampicillin or an antiviral agent such as ribavirin, for example, can be useful for treating a bacterial or viral infection in a selected organ or tissue.

A therapeutic agent also can inhibit or promote the production or activity of a biological molecule, the expression or deficiency of which is associated with the pathology. Thus, a protease inhibitor can be a therapeutic agent that, when linked to an organ homing molecule, can inhibit protease activity at the selected organ or tissue, for example, the pancreas. A gene or functional equivalent thereof such as a cDNA, which can replenish or restore production of a protein in a selected organ or tissue, also can be a therapeutic agent useful for ameliorating the severity of a pathology. A therapeutic agent also can be an antisense nucleic acid molecule, the expression of which inhibits production of a deleterious protein, or can be a nucleic acid molecule encoding a dominant negative protein or a fragment thereof, which can inhibit the activity of a deleterious protein.

### Photodynamic Therapy (PDT) Agents

PDT is a promising cancer treatment that involves the combination of light and a photosensitizer. Each factor is harmless by itself, but when combined together, they can produce lethal reactive oxygen species that kill the tumor cells (Dougherty, T. J. et al. Journal of the National Cancer Institute. 90, 889-905 (1998)). Singlet oxygen (¹O₂) is a powerful, fairly indiscriminate oxidant that reacts with a variety of biological molecules and assemblies. It is generally recognized that ¹O₂ is the key agent of PDT induced tumor necrosis (Niedre, M. et al. Photochemistry & Photobiology. 75, 382-391 (2002)). The diffusion range of ¹O₂ is limited to approximately 45 nm in cellular media (Moan, J. Photochem. Photobiol. 53, 549-553 (1991)). Therefore, the site of the primary generation of O₂ determines which subcellular structures may be accessed and attacked. In other words, if a photosensitizer is preferentially localized in tumor cells, PDT induced cellular damage is highly tumor specific.

Preferred photodynamic therapy agents are porphyrins, porphyrin isomers, and expanded porphyrins.

In embodiments, the photodynamic therapy agent is selected from the group consisting of SiNc-BOA, SiPc-BOA, and pyropheophorbide-cholesterol ester (Pyro-CE).

### Near-Infrared (NIR) Dyes for Fluorescent Imaging and PDT Agents

NIR fluorescent imaging (NIRF) is a non-radioactive, highly sensitive, and inexpensive cancer detection modality (Weissleder, R. et al. Nature Medicine 9, 123-128 (2003), Frangioni, J. V. Current Opinion in Chemical Biology 7, 626-634 (2003)), which permits noninvasive differentiation of tumor and healthy tissue based on differences in their fluorescence. NIR dyes are presently attracting considerable interest as NIRF probes for detection of cancer and as photosensitizers for cancer treatment by PDT. The appeal of NIR dyes resides in the tissue optical properties in the spectral window between 600 nm to 900 nm. For such wavelengths, the tissue absorption coefficients are relatively low; thus, the propagation of light is mainly governed by scattering events, and a penetration depth of several centimeters is attainable. Therefore, the unique capability of NIR dyes enables fluorescent imaging and PDT treatment of subsurface tumors, including breast cancer.

A current limitation of both NIRF and PDT modalities is their lack of sufficient tumor-to-tissue contrast due to the relatively non-specific nature of delivering the dye to the tumor, which has led to false negatives for NIRF and inadequate tumor-to-normal tissue therapeutic ratio for PDT. Hence, agents targeting “cancer signatures”, i.e. molecules that accumulate selectively in cancer cells, are particularly attractive. This invention provides tumor-targeting LBNPs locking NIRF/PDT agents inside the LDL core so that a higher probe/protein molar ratio and tumor specificity is achieved.

### Magnetic Resonance Imaging Agents

It is now well established that MRI is the pre-eminent methodology among the various diagnostic modalities currently available, as it offers a powerful way to map structure and function in soft tissues by sampling the amount, flow, and environment of water protons in vivo. The intrinsic contrast can be augmented by the use of contrast agents. Targeted MRI agents, though extremely attractive conceptually, exist in only a few potentially useful examples. Because of sensitivity limitations, efficient recognition currently requires a very high capacity target like fibrin, which is present in sufficient quantity to be seen with simple targeted Gd chelates, or targets accessible to the blood stream that can be bound with a Gd cluster, polymer or an iron particle. This is presently a very limited target set. Moreover, intracellular MRI imaging is particularly challenging because the minimum concentration of MRI agents required for the MRI detection limit is much higher (˜1 mM) than the extracellular targeting threshold (40 μ1V1) (Aime, S. et al. Journal of Magnetic Resonance Imaging 2002 16(4):394-406, Nunn, A. D. et al. Quarterly Journal of Nuclear Medicine. 1997 41(2):155-62). One notable attempt was reported by Wiener et al. in 1995 (Wiener, E. C. et al. Investigative Radiology. 1997 December, 32(12):748-54). Using a folate-conjugated DTPA-based dendrimer, they achieved uptake by tumor cells which was related to the presence of the folate receptor. They also obtained MRI contrast enhancement of 17% 24 h after injection. The present invention provides LBNPs to deliver both MRI and NIRF/PDT agents.

In certain embodiments of the present invention, the MRI contrast agent is an iron oxide or a lanthanide base, such as gadolinium (Gd³⁺) metal.

### Agents Active in the Nervous System

Drugs that are active on the nervous system can also be delivered via the LBNPs of the present invention. Such drugs include antipsychotics, stimulants, sedatives, anesthetics, opiates, tranquilizers, antidepressants, such as MAO inhibitors, tricyclics and tetracyclics, selective serotonin reuptake inhibitor and burpropion. Drugs that are active in the nervous system also include neuropeptides. Delivery of peptide drugs is limited by their poor bioavailability to the brain due to low metabolic stability, high clearance by the liver and the presence of the blood brain barrier. The LBNPs of the present invention enable the delivery of such drugs to the central nervous system.

### Pharmaceutical Compositions

When administered to a subject, the nanoplatforms of the present invention are administered as a pharmaceutical composition containing, for example, the conjugate and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters.

A pharmaceutically acceptable carrier can contain physiologically acceptable compounds that act, for example, to stabilize or to increase the absorption of the complex. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the composition. The pharmaceutical composition also can contain an agent such as a cancer therapeutic agent or other therapeutic agent as desired.

As noted above, the nanoplatforms of the present invention may be provided in a physiologically or pharmaceutically acceptable carrier, or may be provided in a lyophilized form for subsequent use. The compositions are optionally sterile when intended for parenteral administration or the like, but need not always be sterile when intended for some topical application. Any pharmaceutically acceptable carrier may be used, including but not limited to aqueous carriers. Aqueous carriers for parenteral injections include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

One skilled in the art would know that a pharmaceutical composition containing the conjugates of the present invention can be administered to a subject by various routes including, for example, orally or parenterally, such as intravenously. The composition can be administered by injection or by intubation.

In performing a diagnostic or therapeutic method as disclosed herein, a therapeutically effective amount of a conjugate of the present invention must be administered to the subject. A “therapeuticall effective amount” is the amount of the conjugate that produces a desired effect. An effective amount will depend, for example, on the active agent and on the intended use. For example, a lesser amount of a radiolabeled conjugate can be required for imaging as compared to the amount of the radiolabeled molecule administered for therapeutic purposes, where cell killing is desired. A therapeutically effective amount of a particular conjugate for a specific purpose can be determined using methods well known to those in the art.

In principle, an organ homing molecule as part of a LBNP of the present invention of the invention can have an inherent biological property, such that administration of the molecule provides direct biological effect. For example, an organ homing molecule can be sufficiently similar to a naturally occurring ligand for the target molecule that the organ homing molecule mimics the activity of the natural ligand. Such an organ homing molecule can be useful as a therapeutic agent having the activity of the natural ligand. For example, where the organ homing molecule mimics the activity of a growth factor that binds a receptor expressed by the selected organ or tissue, such as a skin homing molecule that mimics the activity of epidermal growth factor, administration of the organ homing molecule can result in cell proliferation in the organ or tissue. Such inherent biological activity of an organ homing molecule of the invention can be identified by contacting the cells of the selected organ or tissue with the homing molecule and examining the cells for evidence of a biological effect, for example, cell proliferation or, where the inherent activity is a toxic effect, cell death.

In addition, an organ homing molecule as part of the LBNP of the invention can have an inherent activity of binding a particular target molecule such that a corresponding ligand cannot bind the receptor. It is known, for example, that various types of cancer cells metastasize to specific organs or tissues, indicating that the cancer cells express a ligand that binds a target molecule in the organ to which it metastasizes. Thus, administration of a lung homing molecule, for example, to a subject having a tumor that metastasizes to lung, can provide a means to prevent the potentially metastatic cancer cell from becoming established in the lung. In general, however, the organ homing molecules of the invention are particularly useful for targeting a moiety to a selected organ or tissue. Thus, the invention provides methods of treating a pathology in a selected organ or tissue by administering to a subject having the pathology a LBNP of the present invention.

Specific disorders of the lung, for example, can be treated by administering to a subject a LBNP comprising a lung homing molecule and a therapeutic agent. Since a lung homing molecule can localize to the capillaries and alveoli of the lung, disorders associated with these regions are especially amenable to treatment with a conjugate comprising the lung homing molecule. For example, bacterial pneumonia often originates in the alveoli and capillaries of the lung (Rubin and Farber, Pathology 2nd ed., (Lippincott Co., 1994)). Thus, a LBNP with a lung homing molecule and a suitable antibiotic can be administered to a subject to treat the pneumonia via a LBNP of the present invention. Similarly, cystic fibrosis causes pathological lesions in the lung due to a defect in the CFTR. Thus, administration of a LBNP with a lung homing molecule and a nucleic acid molecule encoding the CFTR provides a means for directing the nucleic acid molecule to the lung as an in vivo gene therapy treatment method.

The invention also provides methods of treating a pathology of the skin by administering to a subject having the pathology an LBNP comprising a skin homing molecule and a therapeutic agent. For example, a burn victim can be administered an LBNP comprising a skin homing molecule and an epithelial growth factor or platelet derived growth factor such that the growth factor is localized to the skin where it can accelerate regeneration or repair of the epithelium and underlying dermis. Furthermore, a method of the invention can be useful for treating skin pathologies caused by bacterial infections, particularly infections that spread through the hypodermis and dermis or that are localized in these regions, by administering to a subject a conjugate comprising a skin homing molecule linked to an antibiotic.

The invention also provides methods of treating a pathology of the pancreas by administering to a subject having the pathology an LBNP comprising a pancreas homing molecule and a therapeutic agent. In particular, since a pancreas homing molecule of the invention can localize to the exocrine pancreas, a pathology associated with the exocrine pancreas can be treated and, in some cases, may not adversely affect the endocrine pancreas. A method of the invention can be particularly useful to treat acute pancreatitis, which is an inflammatory condition of the exocrine pancreas caused by secreted proteases damaging the organ. A LBNP comprising a pancreas homing molecule and a protease inhibitor can be used to inhibit the protease mediated destruction of the tissue, thus reducing the severity of the pathology. Appropriate protease inhibitors useful in such a conjugate are those that inhibit enzymes associated with pancreatitis, including, for example, inhibitors of trypsin, chymotrypsin, elastase, carboxypeptidase and pancreatic lipase. A method of the invention also can be used to treat a subject having a pancreatic cancer, for example, ductal adenocarcinoma, by administering to the subject a LBNP comprising a therapeutic agent linked to a molecule that homes to pancreas.

The methods of the invention also can be used to treat a pathology of the eye, particularly the retina, by administering to a subject having the pathology an LBNP comprising a retina homing molecule and a therapeutic agent. For example, proliferative retinopathy is associated with neovascularization of the retina in response to retinal ischemia due, for example, to diabetes. Thus, administration of a conjugate comprising a retina homing molecule linked to a gene that stimulates apoptosis, for example, Bax, can be used to treat the proliferative retinopathy. Similarly, methods of the invention can be used to diagnose or treat prostate, ovary, breast, lymph node, adrenal gland, liver, or gut pathology using the appropriate organ or tissue homing molecules disclosed herein.

The invention further provides methods of delivering a lipophilic compound, diagnostic agent or drug to a subject, target tissue, or organ comprising the steps of preparing a pharmaceutical formulation comprising a lipophilic drug in association with a lipid in an amount sufficient to form a complex with said lipophilic drug according to the methods of the invention and administering a therapeutically effective amount of the pharmaceutical formulation to said target tissue. The pharmaceutical formulation of the invention may be administered intravenously, intraarterially, intranasally such as by aerosol administration, nebulization, inhalation, or insufflation, intratracheally, intra-articularly, orally, transdermally, subcutaneously, or topically.

By “effective” or “therapeutically effective” amount is meant an amount that relieves (to some extent) one or more symptoms of the disease or condition in the patient. Additionally, by “therapeutically effective amount” is meant an amount that returns to normal, either partially or completely, physiological or biochemical parameters associated with or causative of a condition. Additionally, the effective amount may be one sufficient to achieve some other intended purpose, such as delivery of a radioimaging agent or other diagnostic agent to an organ or tissue.

Still further, the present invention provides a method of identifying a selected organ or tissue or diagnosing a pathology in a selected organ or tissue comprising the steps of preparing a pharmaceutical formulation comprising an appropriate targeting moiety and a diagnostic agent in association with a lipid in an amount sufficient to form a particle with said diagnostic agent according to the methods of the invention and administering to a subject a pharmaceutical formulation to said target organ or tissue.

“Diagnostic agent” refers to any agent which may be used in connection with methods for imaging an internal region of a patient and/or diagnosing the presence or absence of a disease in a patient. Exemplary diagnostic agents include, for example, radioactive and fluorescent labels and contrast agents for use in connection with ultrasound imaging, magnetic resonance imaging or computed tomography imaging of a patient. Diagnostic agents may also include any other agents useful in facilitating diagnosis of a disease or other condition in a patient, whether or not imaging methodology is employed.

The present invention also provides a method of treating a subject suffering from a disorder selected from the group consisting of skin cancer, psoriasis, acne, eczema, rosacea, actinic keratosis, seborrheic dermatitis, and congenital keratinization disorders, in which any composition according to the methods of the invention is administered to the subject in need of such treatment by means of topical application.

The LBNPs of the present invention can, as noted above, be used for topical application. Thus the present invention further provides a method of treating one or more conditions of the skin selected from the group consisting of dry skin, photodamaged skin, age spots, aged skin, increasing stratum corneum flexibility, wrinkles, fine lines, actinic blemishes, skin dyschromias, and ichthyosis, comprising applying to the skin having said one or more condition any composition according to the methods of the invention, where the compound to be delivered is a known compound for treating such conditions, and is delivered in its known amount for treating such conditions. The term “topical application”, as used herein, means to apply or spread the compositions of the present invention onto the surface of the skin.

The compound to be delivered in topical compositions of the present invention may comprise skin active ingredients. Non-limiting examples of such skin active ingredients include vitamin B3 compounds such as those described in PCT application WO 97/39733, published Oct. 30, 1997, to Oblong et al., herein incorporated by reference in its entirety; flavonoid compounds; hydroxy acids such as salicylic acid; exfoliation or desquamatory agents such as zwitterionic surfactants; sunscreens such as 2-ethylhexyl-p-methoxycinnamate, 4,4′-t-butyl methoxydibenzoyl-methane, octocrylene, phenyl benzimidazole sulfonic acid; sun-blocks such as zinc oxide and titanium dioxide; anti-inflammatory agents; anti-oxidants/radical scavengers such as tocopherol and esters thereof; metal chelators, especially iron chelators; retinoids such as retinol, retinyl palmitate, retinyl acetate, retinyl propionate, and retinal; N-acetyl-L-cysteine and derivatives thereof; hydroxy acids such as glycolic acid; keto acids such as pyruvic acid; benzofuran derivatives; depilatory agents (e.g., sulfhydryl compounds); skin lightening agents (e.g., arbutin, kojic acid, hydroquinone, ascorbic acid and derivatives such as ascorbyl phosphate salts, placental extract, and the like); anti-cellulite agents (e.g., caffeine, theophylline); moisturizing agents; anti-microbial agents; anti-androgens; and skin protectants. Mixtures of any of the above mentioned skin actives may also be used. A more detailed description of these active ingredients is found in U.S. Pat. No. 5,605,894 to Blank et al. Preferred skin active ingredients include hydroxy acids such as salicylic acid, sunscreen, antioxidants and mixtures thereof. Topical applications can be practiced by applying a composition of the invention in the form of a skin lotion, cream, gel, emulsion, spray, conditioner, cosmetic, lipstick, foundation, nail polish, or the like which is intended to be left on the skin for some esthetic, prophylactic, therapeutic or other benefit.

### Methods of Making the LBNPs of the Invention

Nanoplatform Core Loading

The present invention provides methods of making the LBNPs of the present invention. In general, such LBNPs can be made by first loading the core of a lipoprotein particle with at least one active agent. The nanoparticle can be any of the lipoproteins listed in Table 1. Such lipoproteins include chylomicrons, VLDL, IDL, LDL, or HDL. The invention also provides methods of making an LBNP, wherein the active agent is loaded after the homing molecule is attached to the surface.

In general, there are three ways to incorporate agents into LDL particles. The first method involves the direct affixation of probes to the amino acid residues of the apoprotein of the lipoprotein particle. To date this has been done for only a few radioactive imaging agents with LDL particles (125I, 111In or 68Ga labeled LDL). (Moerlein, S. M., Daugherty, A., Sobel, B. E. & Welch, M. J. Metabolic imaging with gallium-68- and indium-111-labeled low-density lipoprotein. Journal of Nuclear Medicine. 1991 32(2):300-7), and in most cases, the probe/LDL ratio was generally kept low to avoid disrupting the 3-D structure of the recognition protein. Lund-Katz et al. demonstrated that when ˜20% of the Lys residues on the apoB-100 are capped via reductive methylation, binding to the LDLR is essentially abolished. Thus, modification of lysines is to be avoided, even at low labeling ratios, when retention of LDLR binding is essential.

As a second approach, lipid-anchored probes can be incorporated into the LDL phospholipid monolayer via an intercalation mechanism. For example, the LDL can be labeled with ¹¹¹In via a lipid-anchored diethylenetriaminepentaacetic acid (DTPA) chelating agent, as a radiopharmaceutical for tumor localization. (Urizzi, P. et al., International Journal of Cancer. 1997 Jan. 27; 70(3):315-22). These phospholipid intercalating agents might exchange thermodynamically with similar sites on the plasma membranes of cells, thus reducing the specificity for LDLR. Following this approach, the potential of using fluorescent dye-labeled LDL as optical probes for cancer detection was also investigated.

A third method is the LDL reconstitution approach. (Krieger, M. et al. Proceedings of the National Academy of Sciences of the United States of America. 1978 75(10):5052-6.) were the first to report that it is possible to remove more than 99% of core cholesteryl esters from the LDL particle by heptane extraction and replace them with an equivalent amount of exogenous cholesteryl linoleate. The reconstituted LDL (rLDL) particle is essentially identical to native LDL in its ability to bind to LDLR, to be internalized by cells and to be hydrolyzed in lysosomes. Moreover, the cholesterol released from the lysosomal hydrolysis of the rLDL retained its ability to modulate cholesterol metabolism. Since rLDL is internalized preferentially by LDLR, cytotoxic compounds (e.g., doxorubicin (Firestone, R. A. et al., J. Med. Chem. 27, 1037-1043 (1984)) have been delivered to cancer cells using this method and have shown good anti-tumor activity.

In addition to LDL, HDL has been explored as a drug-carrier system for a hydrophobic prodrug of IUdR and for cervical and breast cancer chemotherapy. (Kader et al., J. Control Release 80:29-44 (2002) and Bijsterbosch et al., Biochemistry 33:14073-14080 (1994)). HDL plays a major role in the transport of cholesterol from peripheral tissues to the liver (called ‘reverse cholesterol transport’). (Pieters et al., Biochim Biophys Acta 1225:125-134 (1994)). HDL transports cholesterol to liver cells, where cholesterol is recognized and taken up via specific receptors. Cholesteryl esters within HDL are selectively uptaken by hepatocytes via the scavenger receptor BI(SR-BI). (Acton S. et al., Science 1996; 271: 518-520, Acton S L. et al, Mol Med Today 1999; 5: 518-524).

Lou et al., have reported reconstitution of HDL with aclacinomycin (ACM) that keeps the basic physical and biological binding properties of native HDL and shows a preferential cytotoxicity for SMMC-7721 hepatoma to normal L02 hepatocytes. (Lou et al., World J. Gastroenterol. 11:954-959 (2005)).

A reconstituted rHDL-drug complex can be formed by dispersing a lipid, such as soy phosphatidylcholine, and drug in buffer, for example, 0.01 mol/L pH 8.0 Tris buffer (containing 0.1 mol/L KCl, 1 mmol/L EDTA and 0.02% NaN3) and sonicating using a probe sonicator for, for example, 30 min at room temperature. Then, an HDL apoprotein, such as apoA-I, can be added over a period of, for example, 5 min. Sonication can then be continued for a period of time, for example, 10 min. The preparation can be purified by density gradient centrifugation and exhaustively dialyzed against, for example, 0.15 mol/L NaCl, 1 mmol/L sodium EDTA, 0.02% NaN3, and pH 6.5. After dialysis, the prepared rHDL-ACM can be purified via column chromatography using, for example, SephadexG-25 (1′18 cm) ACM.

Attachment of Cell Surface Receptor Ligand

Following loading of the core of the LBNP with an active agent, the surface of the LBNP is modified to attach a cell surface receptor ligand. Alternatively, the cell surface receptor ligand can also be attached prior to loading the active agent. In some embodiments, the cell surface receptor ligand is covalently bonded to the apoprotein present in the LBNPs of the present invention.

The mature apoB-100 molecule comprises a single polypeptide chain of 4536 amino acid residues. Chemical modification of functional groups in the apoB-100 molecule has shown that the electrostatic interaction of domains containing basic Lys and Arg residues with acidic domains on the LDLR is important to the binding process. (Mahley, R. W. et al., Journal of Biological Chemistry. 1977 Oct. 25; 252(20):7279-87). The involvement of Lys in the LDLR binding process is particularly important. There are two types of Lys residues on the apoB-100 protein; “active” Lys have a pK of about 8.9, while “normal” Lys have a pK of about 10.5. (Lund-Katz, S. et al. Journal of Biological Chemistry. 1988 Sep. 25; 263(27):13831-8). ApoB-100 contains 53 active and 172 normal Lys residues are exposed on the surface of LDL with the remaining 132 Lys residues (a third of total Lys) which are present in apoB-100 being buried and unavailable for reaction.

Effective Lys modifications include reaction of LDL with organic acid anhydrides (acetylation or maleylation) and reaction with aldehydes, such as malondialdehyde. (Brown, M. S. et al., Journal of Supramolecular Structure. 1980; 13(1):67-81). Reductive methylation with formaldehyde and sodium cyanoboronhydride is also an effective Lys capping technique. (Lund-Katz, S. et al., Journal of Biological Chemistry. 1988 Sep. 25; 263(27):13831-8.) Almost all Lys residues exposed on the LDL surface (two third of total Lys: 225) can be capped by these procedures. The fact that Lys residues can be transformed without significant alteration in their pK means that such LDL modification does not alter the conformation of the apoB-100. However, these modifications do impair the ability of apoB-100 on LDL to bind to the LDLR. Lund-Katz et al. demonstrated that when about 20% of the Lys are capped, binding to the LDLR is essentially abolished. The ability of LDL to bind to the LDLR is reduced by 50% when about 8% of the Lys residues are methylated.

Attachment of cell surface receptor ligands to the reconstituted lipoprotein particles of the present invention can occur via standard techniques. For example, the ligand folic acid can be attached to LDL particle via increasing the pH by dialyzing LDL against a buffer, i.e., NaH₂PO₄/H₃BO₃ buffer. Folic acid-N-hydroxysuccinimide ester is then reacted with the LDL particle at, for example, 4° C. for 30 h. Upon completion of the reaction, the mixture is centrifuged to remove any degraded LDL. In the final step, crude LDL-FA can be dialyzed against EDTA buffer to adjust the pH to more acidic to remove unreacted FA.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein is come apparent to those skilled in the art from the foregoing description and accompanying drawings. Such modifications are intended to fall within the scope of the appended claims.

Each reference cited herein is expressly incorporated by reference in its entirety.

### EXAMPLES

### Example 1

### Low Density Lipoprotein Reconstituted Using Pyropheophorbide Cholesterol Oleate

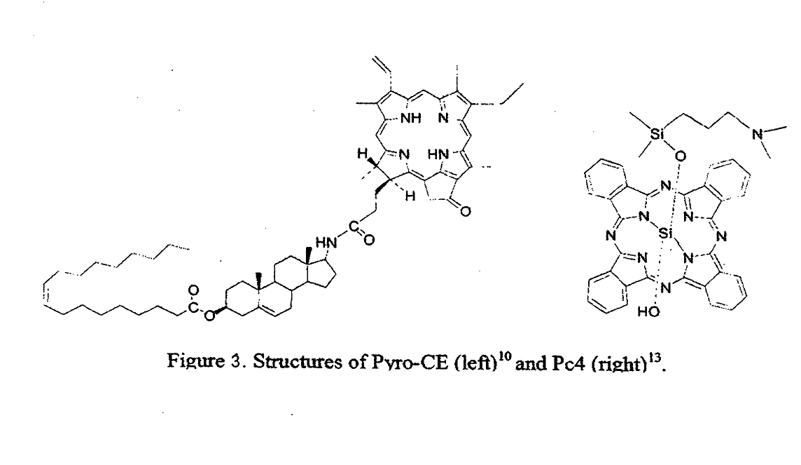

A novel, chlorophyll-based photosensitizer (“PS”) containing anchors that render it compatible with LDL's phospholipid coat and lipophilic core was synthesized (Weissleder, R. & Ntziachristos, V. Nature Medicine 9, 123-128 (2003)). This new dye conjugate, pyropheophorbide cholesterol oleate (Pyro-CE) (FIG. 3), contains an oleate moiety to facilitate LDL reconstitution and a cholesterol moiety to anchor the phospholipid monolayer to prevent probes from leaking. Pyro-CE was incorporated into LDL (r-Pyro-CE-LDL) with a modest PS payload (Pyro-CE:LDL molar ratio <50:1). The reconstitution efficiency of r-Pyro-CE-LDL is 45% as determined by Lowry's method, which is similar to the ˜55% cholesteryl linoleate LDL reconstitution efficiency. (Krieger, M. et al., Proc. Natl. Acad. Sci. USA. 75, 5052-6 (1978)). Laser scanning confocal microscopy studies demonstrated that such an r-LDL based PS was internalized exclusively by LDLR overexpressing human hepatoblastoma G2 (HepG₂) tumor cells and accumulated in intracellular compartments such as endosomes or lysosomes. (Zheng G, et al., Bioconjugate Chem. 13, 392-396 (2002)).

Using LDL Particle to Deliver NIRF/PDT agents to Tumor Cells Via LDLR Pathway

In this section, the following are demonstrated to: 1) synthesize stable and biocompatible NIRF and PDT agents suitable for LDL reconstitution, 2) incorporate large payloads of NIRF/PDT agents into LDL particles, 3) perform quantitative analysis of receptor-binding characteristics of a given tumor cell line, 4) using systematic in vitro and in vivo imaging techniques, to characterize the LDLR-mediated uptake of LDL particles loaded with NIRF/PDT agents in tumor cells, and 5) determine the PDT efficacy in vitro and in vivo. The knowledge obtained from these studies should facilitate designing the LBNP-delivered NIRF/PDT agents.

Synthesis of Neutral, Stable and Lipid-Anchored Dyes for Both NIRF and PDT:

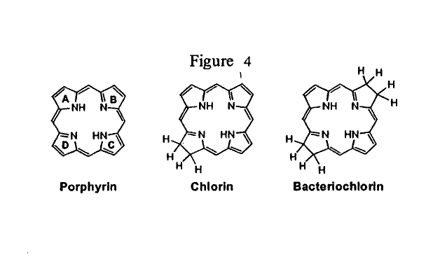

Porphyrins (see FIG. 4) are 18 π-electron aromatic macrocycles that exhibit characteristic optical spectra with a very strong π−π/t* transition around 400 nm (Soret band) and usually four Q bands in the visible region. Two of the peripheral double bonds in opposite pyrrolic rings are cross-conjugated and are not required to maintain aromaticity. Thus, reduction of one or both of these cross-conjugated double bonds maintains much of the aromaticity, but the change in symmetry results in red-shifted Q bands with high extinction coefficients. (Pandey, R. K. & Zheng, G. in The Porphyrin Handbook, Vol. 6. (eds. K. M. Kadish, K. M. Smith & R. Guilard) 157-230 (Academic Press, Boston; 2000)). Generally, the longest wavelength absorption band for porphyrin, chlorin and bacteriochlorin are near 630 nm (ε: 5,000 M⁻¹cm⁻¹), 660 nm (ε: 45,000 M⁻¹cm⁻¹) and 760 nm (ε: 75,000 M⁻¹cm⁻¹), respectively.

Bacteriochlorophyll (BChl), the natural prototype of bacteriochlorin, has several photophysical and chemical characteristics that make it an ideal candidate for PDT. It is a good singlet oxygen producer (Φ_{Δ}˜0.45) and has strong absorption at 780 nm (ε>70,000) near the optimum wavelength for tissue penetration (Henderson, B. W. et al. Journal of Photochemistry & Photobiology. B-Biology. 10, 303-313 (1991)). However, progress in application of naturally occurring bacteriochlorins for PDT has been hampered by the sensitivity of the BChI macrocycle to oxygen (Scheme 1), which results in rapid oxidation to the chlorin state (˜660 nm); thus, the spectroscopic properties of the bacteriochlorins are degraded. Furthermore, if a laser is used to excite the bacteriochlorin in vivo, oxidation may result in the formation of a new chromophore absorbing outside the laser window, thus reducing the PDT efficacy. Preparation of a stable bacteriochlorophyll analog is, therefore, a synthetic challenge. In recent years, many approaches to removing the major points of fragility in the bacteriochlorophyll a molecule have been tried by various investigators. (Kozyrev, A. N. et al., Tetrahedron Letters 37, 6431-6434 (1996), Zilberstein, J. et al., Photochemistry & Photobiology. 73, 257-266 (2001), Chen, Y. et al., Journal of Medicinal Chemistry. 2002 Jan. 17; 45(2):255-8, Fiedor, J. et al., Photochemistry & Photobiology. 2002 August; 76(2):145-52). These include replacing the central metal, magnesium with other metal ions to form stable complexes as well as modifying the isocyclic ring or replacing the phytyl group at the propionyl residue either through trans-esterification or conversion to the corresponding amide derivatives.

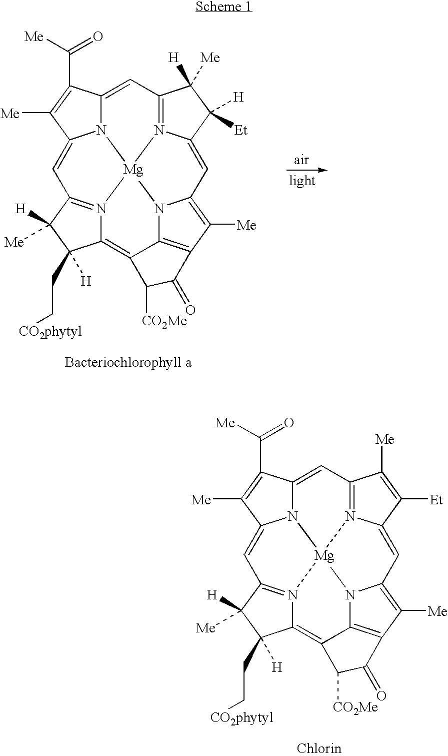

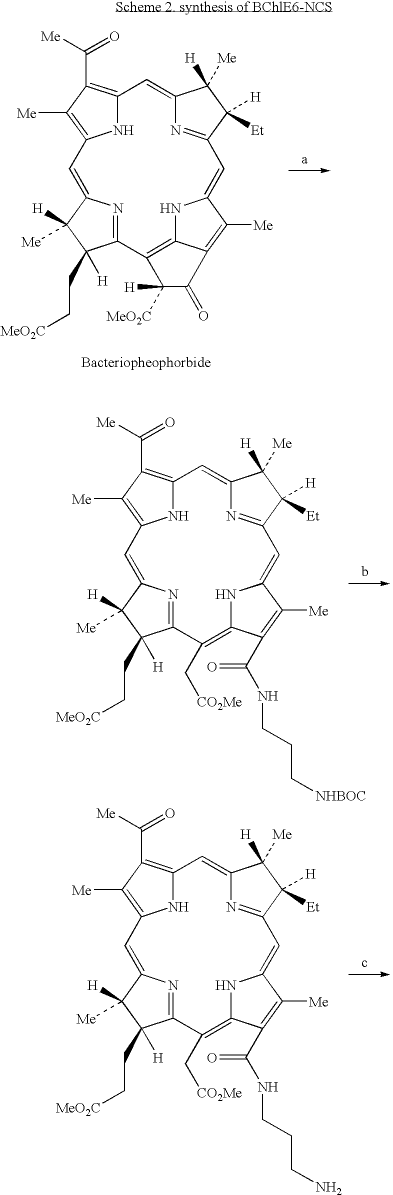

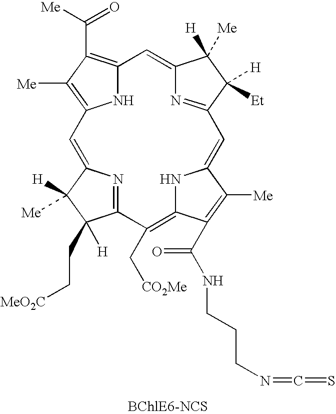

Naturally occurring unstable bacteriochlorophyll a can be converted into stable bacteriochlorins, namely bacteriopurpurin-18 and bacterio-purpurinimide, with remarkable stability and promising in vivo photosensitization efficacy. (Kozyrev, A. N. et al., Tetrahedron Letters 37, 6431-6434 (1996)). In this approach, converting the fused isocyclic ring to a cyclic imide moiety enhanced the stability and solubility of the bacteriochlorophyll analog (BChI). An efficient synthesis of isothiocyanate-containing BChI analogs derived from bacteriochlorin e₆ (BChIE6) (Kim, S. et al., Bioconjugate Chemistry. Submitted) (see Scheme 2) has been developed. Introducing an amine reactive universal linker such as isothiocyanate into the BChI macrocycle allows bioconjugation of these NIR dyes with biologically important molecules such as peptides, metabolites, proteins, and other affinity ligands.

Similar to BChl, chlorophyll a (Chi) is the natural prototype of chlorin. It can be extracted from Spirulina Pacifica in gram quantities and is also the common substrate for the preparation of synthetic BChI. ChI itself absorbs at 666 mm and emits fluorescence at 720 nm in the NIR range. We investigated extensively the chemical modification of natural chlorophyll a and synthesized a series of stable chlorins and bacteriochlorins (FIG. 4). (Zheng, G. et al., Chem. Soc.-Perkin Trans. 1, 3113-3121 (2000), Zheng, G. et al., Zheng, G. et al., Bioorg. Med. Chem. Lett. 10, 123-127 (2000), Chem. Lett., 1119-1120 (1996), Zheng, G. et al., Tetrahedron Lett. 38, 2409-2412 (1997). The knowledge obtained from this study should facilitate designing our LBNP-delivered NIRF/PDT agents.

Synthesis of Lipid-Anchored NIRF/PDT Agents and Their Incorporation into LDL Particles:

To enable a directed and uniform conjugation of the various NIRF/PDT agents, we designed a cholesterol ester containing a primary amine group as the common substrate. (Zheng, G. et al. Tricarbocyanine cholesteryl laurates labeled LDL: new near infrared fluorescent probes (NIRFs) for monitoring tumors and gene therapy of familial hypercholesterolemia. Bioorganic & Medicinal Chemistry Letters. 12, 1485-1488 (2002)). The cholesterol ester moiety is designed to anchor lipids in the LDL core, thereby minimizing non-specific exchange of NIRFs with lipid bilayers on cell membranes. As shown in Scheme 3, we have developed a short and efficient pathway for the synthesis of a stable aminocholesteryl ester in excellent overall yield (60%) from a commercially available cholesteryl amine, 5-androsten-17β-amino-3β-ol (Steraloid Inc., Newport, R.I.). Coupling of this amine to a Pyro and BChi moiety yielded two new NIRF/PDT conjugates in 40% and 85% yields, respectively.

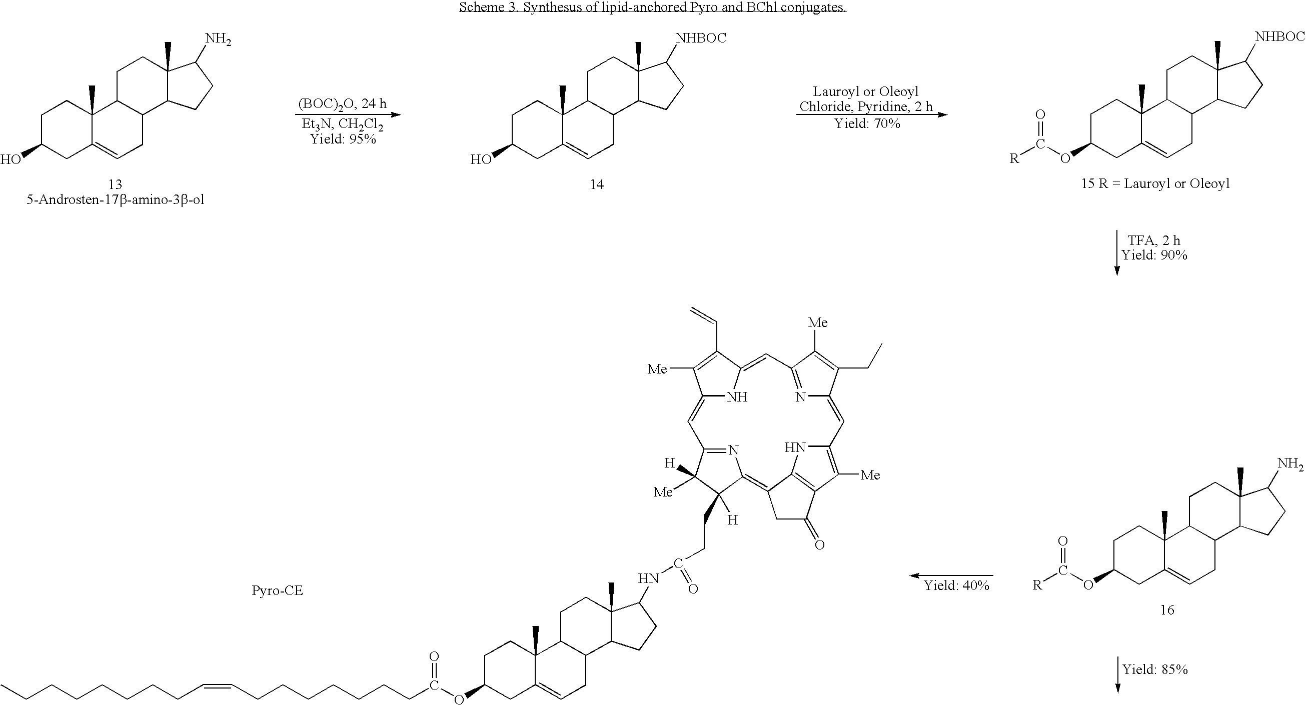

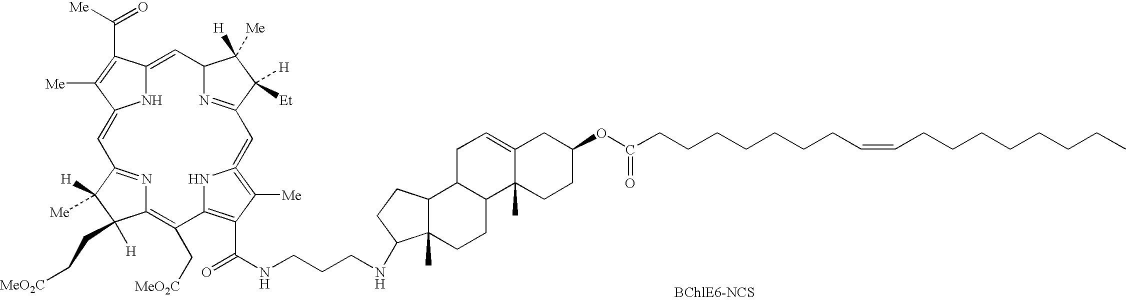

The pyropheophorbide cholesteryl oleate (Pyro-CE) so obtained was then used for reconstituting LDL. (Zheng, G. et al., Bioconjugate Chemistry. 13, 392-396 (2002)). Using a modified Krieger's procedure (Krieger, M., Method Enzymol. 128, 608-613 (1986)), the native cholesteryl esters from the LDL lipid core were extracted and successfully replace with the newly synthesized conjugate. The success of reconstitution was determined by a protein recovery assay following Lowry's method. The pyropheophorbide cholesteryl oleate reconstituted LDL, r-(Pyro-CE)-LDL, yielded a 45% protein recovery comparable to the published value of 48% protein recovery (Krieger, M., Method Enzymol. 128, 608-613 (1986)) for other hydrophobic molecules. The probe to LDL molar ratio was 50:1.

Demonstration of the Selective Delivery of These Modified LDL Particles In Vitro and In Vivo:

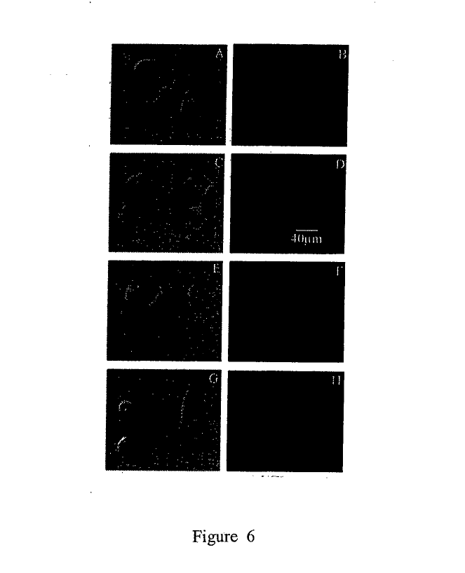

To visualize LDLR-mediated internalization of these NIRF/PDT agents into tumor cells, we performed laser scanning confocal microscopy studies on r-(Pyro-CE)-LDL in HepG₂ tumor cells. Four sets of experiments were performed on a Leica TCS SPII laser scanning confocal microscope (Heidelberg, Germany). FIG. 6 shows the confocal microscopic images of HepG₂ cells incubated with/without fluorescent probe (B, D, F, H) as well as corresponding bright field images (A, C, E, G). In a control experiment, HepG₂ cells were incubated with native LDL but without the photosensitizer at 37° C. for 3 h to determine any possible background fluorescence. As expected, no fluorescence signal was detected without the photosensitizer (FIG. 6B). After incubation with 20 μg/mL of r-(Pyro-CE)-LDL, intense fluorescence signal was observed distributing throughout the whole cell except for the nucleus, presumably in intracellular compartments such as endosomes or lysosomes (FIG. 6D). To determine the specificity of this LDL based photosensitizer toward LDL receptors, HepG₂ cells were incubated with either a 25-fold excess of unlabeled LDL in addition to the 20 μg/mL r-(Pyro-CE)-LDL or with the same amount of Pyro-CE but without reconstitution of this agent into LDL. No fluorescence could be observed in both experiments (FIG. 6F, 6H), which clearly demonstrates specific binding of the reconstituted LDL to the LDLR.

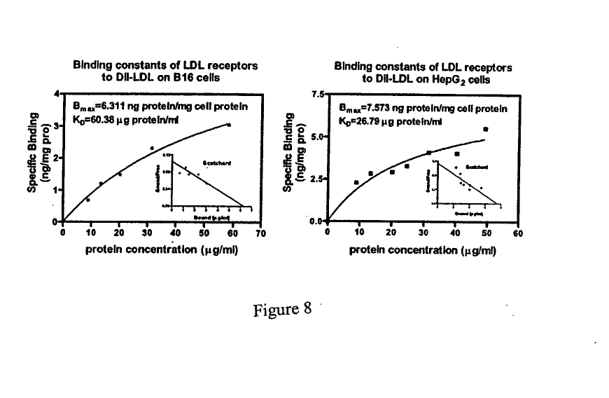

To further confirm the selective delivery of these new NIRF/PDTs to tumors in vivo, a low-temperature 3D fluorescence imagers (Quistorff, B. et al., Analytical Biochemistry. 148, 389-400 (1985), Gu, Y. Q. et al., Rev. Sci. Instruments 73, 172-178 (2002)) was used on an LDLR overexpressing HepG₂ tumor xenograft model. FIG. 6 shows fluorescence images of HepG₂ tumors following intravenous administration of r-(Pryo-CE)-LDL. As shown in this figure, the strong fluorescent signal (red region of the image) of this agent was detected only inside the tumor tissue, demonstrating that r-(Pryo-CE)-LDL was selectively internalized by the tumor. Meanwhile, the high fluorescence intensity in tumor tissue demonstrates the high sensitivity of this new NIRF. In contrast to the HepG₂ tumors, B16 tumors exhibit significantly less fluorescent signals inside the tumor, probably due to three factors: 1) there is a large necrotic area in the center of the tumor (verified by histopathological examination, data not shown); 2) a large amount of melanin might significantly hinder the fluorescence measurement of the B16 tumor; and 3) as indicated by Scatchard analysis (See FIG. 8), the binding affinity to LDL receptors in HepG₂ cells is much higher than that of B16 cells (Li, H. et al. Optical Imaging of Tumors Using Carbocyanine Labeled LDL. Acad. Rad. 11, 669-677 (2004)). Therefore, it is not surprising that the HepG₂ tumor exhibits much more fluorescent signal than the B16 melanoma.

### Example 2

### Low-Density Lipoprotein Reconstituted Using Tetra-t-Butyl Silicon Phthalacyanine Bisoleate (tBu)₄SiPcBOA

To reduce the dose for efficient cancer detection and treatment, it is necessary to maximize the NIR/PDT agent payload for each LDL particle. Thus, a novel strategy to improve LDL's probe payload based on new NIR dyes derived from metallated phthalocyanine (Pc) has been designed. Pc dyes are neutral, porphyrin-like compounds which absorb strongly above 680 nm (within the NIR range of 650-900 nm). They are well-known photosensitizers for PDT (Hasrat Ali and Johan E. van Lier. Metal Complexes as Photo- and Radiosensitizers Chem. Rev. 99, 2379-2450 (1999)) and in general, are much more stable photochemically and photophysically than corresponding porphyrin analogs. Silicon phthalocyanines (SiPc) are of interest for the following reasons: 1) Pc4, a SiPc analog, is currently under PDT cancer clinical trials at the National Cancer Institute (M Egorin et al., Cancer Chemother Pharmacol 44, 283-294 (1999)); 2) the central silicon atom of SiPc allows axial coordination of two bulky ligands on each side of the Pc ring to prevent stack aggregation usually encountered in solution for the planar molecular structure (Farren C, FitzGerald S, Beeby A, Bryce M R. The first genuine observation of fluorescent mononuclear phthalocyanine aggregates. Chem Commun 21, 572-3 (2002)). Such aggregation presumably is the major limiting factor for achieving high probe/LDL payload; 3) since a bent or a branched fatty acid are required for successful LDL reconstitution (Monty Krieger. Reconstitution of hydrophobic core of low-density lipoprotein. Method Enzymol 128, 608-13 (1986)), introducing two oleate moieties via the axial coordination may improve the LDL reconstitution efficiency. In addition, the aggregation phenomenon is known to be responsible for a decrease in the triplet lifetime caused by increased internal conversion, thus leading to less cytotoxic singlet oxygen production. Therefore, eliminating the aggregation effect may help to retain the maximum singlet oxygen production by a Pc-based PS.

In this example, the design and synthesis of tetra-t-butyl silicon phthalocyanine bisoleate, (tBu)₄SiPcBOA is described, and detail its highly efficient LDL reconstitution. Additionally, we characterize the payload and size of the resulting LDL nanoparticles, and demonstrate the in vitro validation for r-SiPcBOA-LDL as a LDLR-specific optical imaging and PDT agent.

### Materials and Methods

Materials: UV-visible and fluorescence spectra were recorded on a Perkin-Elmer Lambda 2 spectrophotometer and LS50B spectrofluorometer, respectively. ¹H NMR spectra were recorded on a Bruker 500 MHz instrument. Mass spectrometry analyses were performed at the Mass Spectrometry Facility of the Department of Chemistry, University of Pennsylvania. All chemicals and reagents were purchased from Aldrich. When necessary, solvents were dried before use. For TLC, EM Science TLC plates (silica gel 60 F₂₅₄) were used.

Synthesis of Bisoleate Conjugate of Silicon Tetra-tert-butyl-phthalocyanine, (tBu)₄SiPcBOA: A suspension of silicon tetra-butyl-phthalocyanine dihydroxide (200 mg, 0.25 mmol) in 20 mL of 2-picoline was mixed with the oleoyl chloride (300 mg, 1.00 mmol) and stirred under argon for 2 h. The 4-dimethylaminopyridine (376 mg, 3.08 mmol) was added to the mixture portion wise, which was kept well stirred under argon for an additional 36 h at 60° C. Upon completion, the solvent was evaporated under reduced pressure, and the product was purified by column chromatography (silica gel-hexane: CH₂Cl₂=1:1) to yield the desired conjugate (130 mg, 0.098 mmol, 39.1%). UV-vis [nm (ε) in CH₂Cl₂]: 362 (1.39×10⁵), 620 (5.32×10⁴), 658 (4.58×10⁴), 691 (2.54×10⁵); Emission λₘₐₓ in CH₂Cl₂ (excitation wavelength 680 nm): 697 nm. ESI-MS calculated for C₈₄H₁₁₄N₈O₄Si: 1327.94, found: 1327.96 (M+); HRMS calculated for C₈₄H₁₁₄N₈O₄Si+Na: 1349.8630, found: 1349.8643. ¹H NMR (CDCl₃, δppm): 9.54-9.70 (m, 8H, Aromatic H), 8.42 (m, 4H, Aromatic H), 5.25 (2 m, each 2H, from oleoyl vinyl H), 1.67-2.01 (m, 52H, from oleoyl chain H), 1.21-1.28 (m, 36H, Boc H), 0.90 (m, 4H, from oleoyl chain), 0.85 (t, 6H, from oleoyl chain terminal CH₃).

LDL Reconstitution and Characterization: LDL, purchased from Dr. Lund-Katz' lab at the Children's Hospital of Philadelphia (Philadelphia, Pa.), was isolated from fresh plasma of healthy donors by sequential ultracentrifugation as described previously. LDL reconstitution with (tBu)₄SiPcBOA was performed following a minor modification of the method of Krieger et al. Briefly, LDL (1.9 mg) was lyophilized with 25 mg starch, and then extracted three times with 5 mL of heptane at −5° C. Following aspiration of the last heptane extract, 6 mg of (tBu)₄SiPcBOA was added in 200 μL of benzene. After 90 min at 4° C., benzene and any residual heptane were removed under a stream of N₂ in an ice salt bath for about 45 min. The r-SiPcBOA-LDL was solubilized in 10 mM Tricine, pH 8.2, at 4° C. for 24 h. Starch was removed from the solution by a low-speed centrifugation (500×g) and followed by a 20 min centrifugation (6000×g). The reconstituted LDL was stored under an inert gas at 4° C. Similarly, r-SiPcBOA-AcLDL was also prepared from (tBu)₄SiPc-BOA and acetylated LDL (AcLDL, Biomedical Technologies, Inc.). The protein content of the specimen was determined by the Lowry method. The absorption spectrum of (tBu)₄SiPc-BOA was measured after extraction with a chloroform and methanol mixture (2:1), and probe concentration was calculated based on the following formula: C=(A/ε)×D, where C is the concentration of the probe, A is the O.D. value, ε is the extinction coefficient, and D is the dilution fold. Probe/protein molar ratio was calculated using the molecular mass of the ApoB-100 protein (514 kDa) knowing that one LDL particle contains only one ApoB-100.

Cell Preparations and Animal Tumor Model: HepG₂ tumor cells, which were obtained from Dr. Theo van Berkel's laboratory from the University of Leiden in the Netherlands, were cultured in Dulbecco's modified Eagle's medium (DMEM) supplemented with 10% fetal bovine serum (FBS), 2 mM L-glutamine, 10 mM HEPES, with 100 U/mL penicillin G sodium and 100 μg/mL streptomycin sulfate. Cells were grown at 37° C. in an atmosphere of 5% CO₂ in a humidified incubator.

Confocal Microscopy Studies: For confocal microscopy studies, HepG₂ cells were grown in 4-well Lab-Tek chamber slides (Naperville, Ill.) at a density of 40,000 cells/well. Experiments were started, after two quick washes with pre-incubation medium (medium with 0.8% (w/v) BSA instead of FBS), by the addition of pre-incubation medium containing the indicated amounts of r-SiPcBOA-LDL/AcLDL and/or unlabeled LDL. After a 4 h incubation at 37° C., the cells were washed three times with ice-cold PBS and fixed for 15 minutes with 3% formaldehyde in PBS at room temperature. Then the chamber slides were mounted and sealed for confocal microscopy analysis. Confocal microscopy was performed with a Leica TCS SPII laser scanning confocal microscope (Heidelberg, Germany). Filter settings were 633 nm for excitation and 638-800 nm for emission.

Electron Microscopy Studies: Five microliters of the reconstituted LDL suspension were placed on carbon-coated 200 mesh copper grids and allowed to stand for 5 min. Excess sample was wicked off with lens paper and 2% Saturated Aqueous Uranyl Acetate was applied to the grid in 5 consecutive drops within 20 seconds. The stain was then drained off with filter paper and the grid was air dried. Digital images were taken using JEOL JEM 1010 electron microscope at 80 kv using AMT 12-HR software aided by Hamamatsu CCD Camera. All related supplies were purchased at Electron Microscopy Sciences (Fort Washington, Pa.).

In Vitro PDT Studies Using r-SiPcBOA-LDL as a Photosensitizer: Flasks containing approximately 2×10⁶ HepG₂ cells were incubated for 5 h at 37° C. in pre-incubation medium with no drug, 10 μg/mL or 50 μg/mL r-SiPcBOA-LDL. Cells were washed with 10 mL HBSS and subsequently incubated for 30 minutes with fresh pre-incubation medium. Cells were again washed with HBSS, collected and resuspended at a concentration of 1×10⁶ cells/mL. Cell suspensions exposed to either drug concentration were then transferred to a 60×15 mm dish and treated with a fluence rate of 5 mW/cm² and a total fluence of 5 J/cm² (1,000 seconds) delivered using a KTP Yag pumped dye module (Laser Scope, San Jose, Calif.) tuned to produce 680 nm of light. Additionally, controls were prepared containing 50 μg/mL r-SiPcBOA-LDL with no light dose exposure, no drug with a 5 J/cm² light dose exposure, or neither drug nor light exposure. Dishes (100×20 mm) were plated in triplicate and placed in a 37° C. incubator (5% CO₂) for 8 days. Following incubation, dishes were rinsed with PBS and allowed to air dry. Subsequently, cells were stained with methylene blue and the colonies counted.

Statistics: Using the statistical package Stata, a square root transformation was performed, followed by an ANOVA. Subsequently, individual t-tests were performed in which drug alone and light alone control groups, as well as, drug and light treatments were compared against the untreated control group.

### Results and Discussion

Design and Synthesis of (tBu)₄SiPcBOA: The aim of this work is to improve the labeling (reconstitution) efficiency of LDL-based PS for achieving high probe to protein payload. To achieve this aim, PS should be neutral, highly soluble in non-polar solvent, have minimal aggregation and contain a suitable linker for conjugation to a lipid anchor to prevent the dye from dissociating from LDL and nonspecifically binding to phospholipid bilayers on cellular membranes. For these purposes, we designed a new PS for LDL reconstitution based on SiPc. Because Si coordination allows the binding of two axial oleate ligands, these ligands will then create steric hindrance on each side of the Pc ring thereby limiting stack aggregation. Therefore, we anticipate a large increase in the PS payload of LDL. Moreover, since aggregation is responsible for a decrease in the triplet lifetime caused by increased internal conversion, we also expect that this design will lead to high cytotoxic singlet oxygen production.

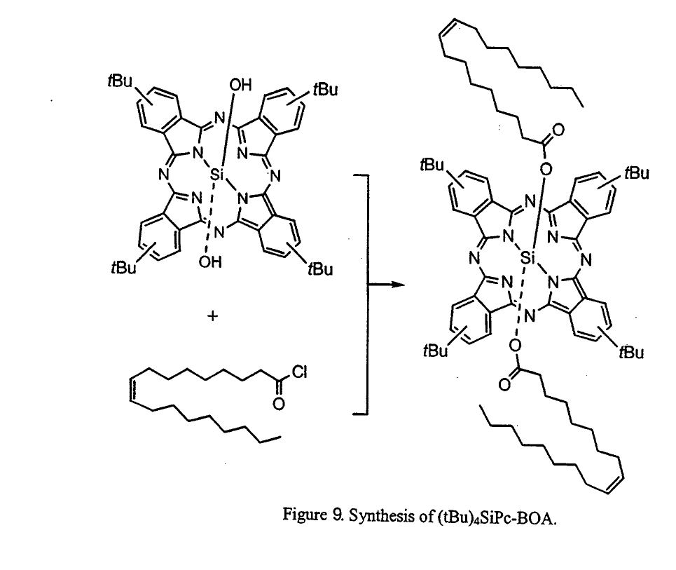

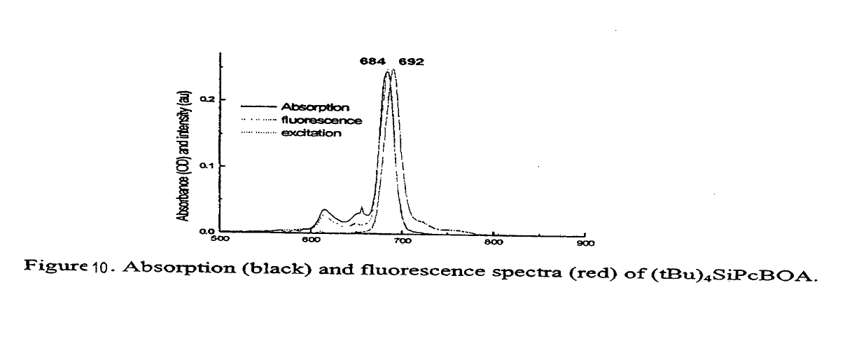

To synthesize bisoleate-anchored SiPc, commercially available silicon tetra-tertbutylphthalocyanine dihydroxide, (tBu)₄SiPc(OH)₂, was conjugated at the axial position with oleoyl chloride in the presence of 4-dimethylaminopyridine and 2-picoline. The desired conjugate, (tBu)₄SiPcBOA, was obtained in 40% yield. This efficient synthetic pathway is depicted in FIG. 9. The structure of this compound was confirmed by ¹H NMR and high resolution mass spectroscopy analysis. FIG. 10 shows the absorption and fluorescence spectra of this new compound. It has a very intense absorption at 684 nm and emission at 692 nm, both within the NIR range.

As shown in FIG. 9, this method has several distinct advantages. Firstly, the reaction condition is very mild. It can be carried out in weak base (picoline, dimethylaminopyridine) at warm temperature (<60° C.) instead of at 150° C. in a much stronger base (sodium alcoholate), as is commonly used for the preparation of Pc derivatives. Secondly, the starting material, (tBu)₄SiPc(OH)₂, is commercially available and it consists of four lipophilic and bulky t-butyl groups at the peripheral position of the Pc macrocycle, further increasing its lipophilicity. Finally, the bisoleate anchor (BOA) is known to strongly associate with the lipid membrane, a characteristic similar to that of the cholesterol moiety. Therefore, for Pc LDL reconstitution, we expect that the bisoleate anchor is an enhancement over the corresponding cholesterol oleate moiety.

LDL Reconstitution and Characterization: Protein recovery determined by the Lowry method (Krieger, M., Goldstein, J. L. & Brown, M. S. Receptor-mediated uptake of low density lipoprotein reconstituted with 25-hydroxycholesteryl oleate suppresses 3-hydroxy-3-methylglutaryl-coenzyme A reductase and inhibits growth of human fibroblasts. Proceedings of the National Academy of Sciences of the United States of America. 1978 October; 75(10):5052-6) is an excellent assay for evaluating the success of the reconstitution. Fifty-five to seventy percent protein recovery was observed for both r-SiPcBOA-LDL and r-SiPcBOA-AcLDL, which is better than that observed for r-Pyro-CE-LDL (Li, L. et al. A novel antiangiogenesis therapy using an integrin antagonist or anti-Flk-1 antibody coated 90Y-labeled nanoparticles. International Journal of Radiation Oncology, Biology, Physics. 2004 Mar. 15; 58(4):1215-27). The absorption spectrum for the recovered (tBu)₄SiPcBOA after LDL reconstitution is the same as it was before the reconstitution, indicating absorbance measurement can serve as the basis for calculating the (tBu)₄SiPcBOA concentration in the reconstituted LDL (payload). It was found that ˜3000-3500 (tBu)₄SiPc-BOA molecules were reconstituted into one LDL molecule core. Compared to the 50:1 probe:protein ratio for r-Pyro-CE-LDL we prepared previously, the new probe design reported here improved probe payload on each LDL nanoparticle by 60 fold.

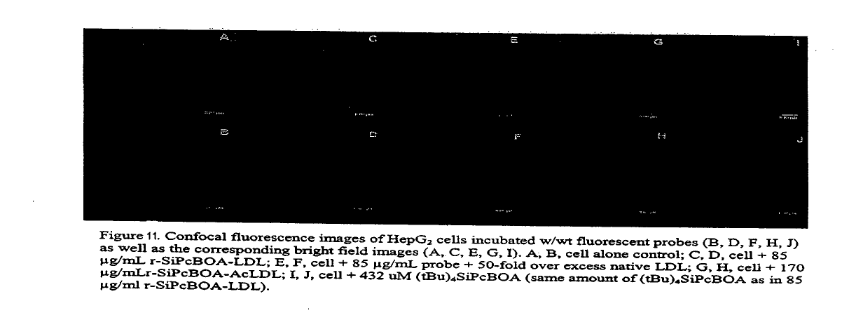

Confocal Microscopy Studies of the LDLR-Specific Uptake: To visualize LDLR-mediated internalization of r-SiPcBOA-LDL, we performed laser scanning confocal microscopy studies on HepG₂ tumor cells. FIG. 11 shows the confocal fluorescent images of HepG₂ cells incubated with/without fluorescent probe (B, D, F, H, J) as well as corresponding bright field images (A, C, E, G, I). FIG. 11A, and B depict images of cell alone, providing values for the fluorescence of the cells. When cells were incubated with 85 μg/mL r-SiPcBOA-LDL at 37° C. for 4 h, the fluorescence signal appears to be localized in the cytoplasm (FIG. 11C, D). To determine the specificity of this r-SiPcBOA-LDL toward LDLR, three sets of control experiments were performed. When HepG₂ cells were incubated with 85 μg/mL r-SiPcBOA-LDL plus 50-fold excess of unlabeled native LDL, complete fluorescence inhibition was observed (FIG. 11E, F). When 170 μg/mL r-SiPcBOA-AcLDL was incubated with HepG₂ cells, despite the fact that the fluorophore concentration doubled, no fluorescence was observed. This is consistent with the inability of Ac-LDL to target LDLR (FIG. 11G, H). FIG. 11I, J show that incubation with the same amount of (tBu)₄SiPcBOA alone did not lead to any observable fluorescence, indicating no internalization occurred. Collectively, above experiments indicate that r-SiPcBOA-LDL was internalized into HepG₂ tumor cells specifically via the LDL receptor pathway.

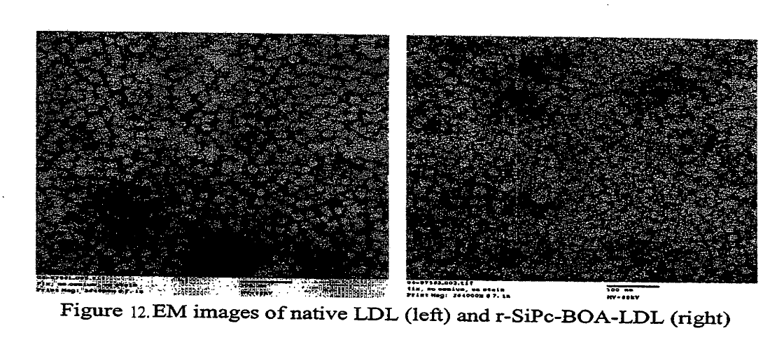

Electron Microscopy Studies: A light scattering size scanner was originally used to measure the size of the SiPc-BOA reconstituted LDL particle. However, we found that Pc absorption interferes with the laser wavelength used by the scanner, therefore electron microscopy was used to directly visualize the LDL particles. As shown in FIG. 12, the mean particle size of r-SiPcBOA-LDL was 23.2±4.6 nm (n=30), which is about the same size as native LDL (20±2.7 nm, n=37).

In Vitro PDT Studies (Clonogenic Assay):

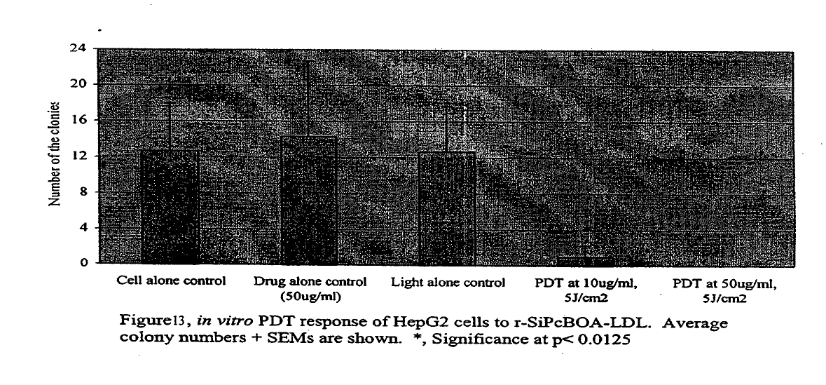

FIG. 13 shows the in vitro PDT response of HepG₂ cells to r-SiPcBOA-LDL using the clonogenic assay. Based upon the results of an ANOVA following a square root transformation, (F(4,10)=29.16 and p<0.0001), individual t-tests were performed. To adjust for multiple comparisons, significance was determined at p<0.0125. The first three columns plot the average number of cell colonies for cell alone, PS drug alone (501.1 g/mL) and light alone control groups, which indicate separate drug and light is not toxic to the cells. When cells were incubated with 10 μg/mL of PS and treated with 5 J/cm² light, 8% of the cells survived (the fourth column in FIG. 13, p<0.0125) compared to the untreated control groups. At a PS dose of 50 μg/mL, none of the cells survived treatment with the same light conditions (the fifth column in FIG. 13, p<0.0125). The data suggest that r-SiPcBOA-LDL can be used as an effective PDT agent.

In conclusion, a new photosensitizer, (tBu)₄SiPc-BOA, was synthesized and successfully reconstituted into the LDL lipid core with very high payloads (3000-3500 probe per LDL molecule), and such payload had no effect on the mean particle size of the LDL nanoparticles. It was found that r-SiPcBOA-LDL′ internalization into HepG₂ tumor cells was exclusively mediated by LDLR as indicated by laser scanning confocal microscopy Moreover, the clonogenic assay demonstrated that r-SiPcBOA-LDL is an effective PDT agent for LDLR overexpressing HepG₂ tumor cells. These data demonstrate that r-SiPcBOA-LDL can be used as a targeted NM optical imaging and PDT agent for cancers overexpressing LDLR.

### Example 3

### Demonstration of the Selective Destruction of Tumor by Using LDLR-Targeted r-(Pryo-CE)-LDL and r-(SiPc-BOA)-LDL as PDT Agents

Synthesis of Lipid-Anchored Phthalocyanine and Naphtha/ocyanine Dyes for LDL Reconstitution: As described above, attempts to model the efficiency of NIRF/PDT probe reconstitution into LDL with Pyro-CE yielded a 50:1 probe to protein molar ratio. This ratio needs to be improved in order to maximize the NIRF/PDT payload for LDL particles. Recently, a new strategy to improve LDL reconstitution efficiency has been designed based on new types of NIR dyes derived from phthalocyanine (Pc) and naphthalocyanine (Nc). Pc and Nc dyes are neutral, porphyrin-like compounds that are well-known PDT agents but so far have not been explored as NIRF probes for tumor imaging. Compared to indocyanine green (ICG), the only FDA approved NIRF probe, they have similar molar absorption (200,000 at 770 nm) but have better photobleaching stabilities (photobleaching quantum yield: 5.0×10⁻⁷ vs 1.7×10⁻⁶), higher fluorescence quantum yield (0.25 vs 0.15) and longer fluorescent lifetimes (2.92 vs 0.76 ns)⁵⁵. The synthesis of both tetra-t-butyl silicon phthalocyanine bisoleate (SiPc-BOA) (Ex: 684 nm) and tetra-t-butyl silicon naphthalocyanine bisoleate (SiNc-BOA) (Ex: 782 nm) (see FIG. 9) has been achieved. These two compounds were designed on the basis of the following considerations. First, because Pc and Nc dyes are notoriously insoluble, four lipophilic t-butyl groups were incorporated at the symmetrical peripheral positions to improve the lipophilicity and to prevent the aggregation of the fluorophore macrocycle. Secondly, two oleate moieties were attached to the central silicon atom at the axial position. Using a bisoleate lipid anchor in place of the cholesterol oleate moiety in Pyro-CE and BChI-CE has several advantages, including preventing aggregation, improving LDL reconstitution via tighter binding to the phospholipid monolayer and allowing one step direct coupling for an efficient synthesis. Indeed, we drastically improved the probe to protein ratio for LDL labeling from 50:1 for reconstituted Pyro-CE-LDL (r-Pyro-CE-LDL), which was described in the preliminary data of the original proposal, to 200:1 for reconstituted SiNc-BOA-LDL (r-SiNc-BOA-LDL) and 500:1 for reconstituted SiPc-BOA-LDL (r-SiPc-BOA-LDL).

In Vivo Characterization of r-SiNc-BOA-LDL:

Using these new NIRF/PDT agents, in vitro and in vivo imaging and PDT studies were performed. In vivo tumor absorption of r-SiNc-BOA-LDL (140 μM, 204 L, i.v. tail-vein, bolus injection) by HepG₂ tumor was measured with a two-channel I&Q spectrometer. The results showed that the probe slowly accumulated in tumor tissues reaching a plateau level at about 2 h. There is no significant change in the uptake by muscle. This clearly demonstrates that large payloads of NIRF/PDT agents can be selectively delivered via LDL nanoparticles to tumor cells overexpressing LDLR.

In Vitro PDT Studies for r-SiPc-BOA-LDL:

Preliminary PDT in vitro studies were also performed to demonstrate that these LDL-transported NIRF/PDT agents exhibit selective PDT efficacy against LDLR-overexpressing tumor cells. HepG2 cells were incubated for 5 h with either no photsensitizer, 50 μg/mL or 10 μg/mL r-SiPc-BOA in DMEM containing 0.8% BSA, 2 mM L-glutamine, 100 U/ml pen-strep, and 10 mM HEPES. Cells were then collected and resuspended at 10 cells/mL. HepG2 cells exposed to 50 ug/mL r-SiPc, or cells incubated with 10 ug/mL r-SiPc-BOA were treated with either 1 J/cm² or 5 J/cm². A light alone control treated with 5 J/cm² was run. Additionally, untreated cells and a drug alone control (50 μg/mL) were plated. PDT parameters included a fluence rate of 5 mW/cm² and a wavelength of 680 nm. Cells were plated in DMEM supplemented with 10% FBS, 2 mM L-glutamine, 10 mM HEPES and 100 U/ml pen-strep. and incubated at 37° C. with 5% CO₂ for approximately two weeks. At a light dose of 5 J/cm², 50 μg/ml r-SiPc-BOA-LDL significantly reduced survival compared to 10 μg/ml r-SiPc-BOA-LDL (p<0.001) (FIG. 12). Additionally, exposure of cells to 50 μg/ml r-SiPc-BOA-LDL at a light dose of 5 J/cm² significantly reduced cell survival (p<0.001) compared to a 1 J/cm² light dose treatment.

Further experimentation revealed that exposure of HepG₂ cells to the potential PDT agent r-SiPc-BOA-LDL at a concentration of 50 μg/ml resulted in a significantly lower survival fraction than cells exposed to either 50 μg/ml r-SiPc-BOA acetylated LDL (p<0.001) or 50 μg/ml dye alone (p<0.001) at identical light doses.

A “point treatment” protocol was used to evaluate PDT response of r-Pyro-CE-LDL. First, instead of irradiating the whole tumor (normally 5-10 mm), we used a special thin optical fiber with a diameter of 1 mm to create a treatment spot of 0.96 mm in diameter. The light field was fixed in position at the center of the tumor by mounting the fiber in the center of a circular plate that was glued to the anesthetized animal. The light was delivered at a fluence rate of 150 mW/cm² to a total dose of 300 J/cm². After light treatment, the mice were rapidly frozen and kept in liquid nitrogen for scanning of oxidized flavoprotein (Fp) and NADH fluorescence. As r-Pyro-CE-LDL-induced PDT resulting in a highly oxidized state of tumor mitochondria. This was determined from the redox ratio changes derived from Fp and NADH fluorescence signals observed using Cryo-imager. This result indicates that redox ratio imaging can be used to monitor PDT response.

### Example 4

### LBNP Targeting at Ovarian Cancers

High expression levels of folate receptors have been shown to occur in many ovarian cancers (Bagnoli M. et al., Gynecologic Oncology 2003 January; 88(1 Pt 2):S140-4, Konda S D, Magma 2001 May; 12(2-3):104-13). Therefore, one or more folic acid moieties (1 to 10) to the lysine residues of the LDL apoB-100 proteins followed by capping the remaining lysine residues on the LDLr binding domain to block LDLr binding. Thus, the LBNP will target the high-affinity folate receptors. LDL reconstitution and LDL intercalation is utilized to add large payloads of diagnostic and therapeutic agents.

### Example 5

### LBNP Targeting at Tumor Vasculatures

Vascular targeting offers therapeutic promise for the delivery of drugs (Arap W. et al., Science 1998 Jan. 16; 279(5349):377-80) and radionuclides (Sipkins D A. et al., Nature Medicine 1998 May; 4(5):623-6). Moreover, targeting of genes to specific blood vessels may provide complementary approaches to disrupt or induce the growth of new blood vessels in various disease states. During vascular remodeling and angiogenesis, endothelial cells show increased expression of several cell surface molecules that potentiate cell invasion and proliferation (Yancopoulos G D. et al., Cell 1998 May 29; 93(5):661-4). One such molecule is the integrin αᵥβ₃, which is preferentially expressed in angiogenic endothelium (Brooks P. C. et al., Cell 1994 Dec. 30; 79(7):1157-64). Therefore, we plan to attach one or more small αᵥβ₃ ligands (1 to 10) to the lysine residues of the apoB-100 protein of the LDL followed by capping the remaining lysine residues on the LDL receptor binding domain to prevent LDL receptor binding. Such LBNP will deliver large payloads of diagnostic and therapeutic agents to tumor vasculature.

### Example 6

### Using LDL Particle to Deliver Gadolinium (Gd)-MRI Agents to Tumor Cells via LDLR Pathway

In this Example, Gd-labelled LDL is used as a selective MRI contrast agent to visualize tumors over expressing LDLR.

Synthesis of Gd-DTPA-Bis(stearylamide)-LDL:

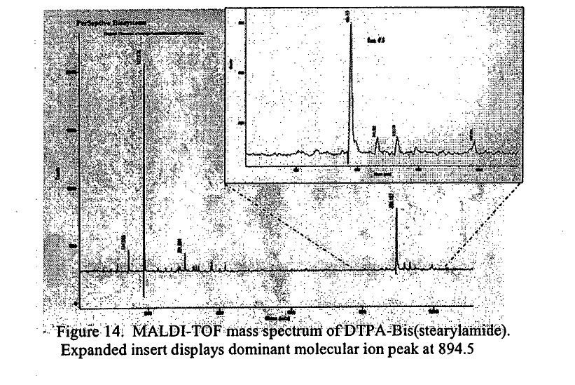

The lipophilic chelate, DTPABis(stearylamide) (DTPA)-SA (FIG. 13) was synthesized from the starting materials, DTPA and stearylamine. This was accomplished by methods similar to (Jasanada, F. et al. Indium-111 labeling of low density lipoproteins with the DTPA-bis(stearylamide): evaluation as a potential radiopharmaceutical for tumor localization. Bioconjugate Chemistry. 1996 January-February; 7(1):72-81) (see methods). The product was obtained in a yield of 48%. The purity of the product was then subsequently checked with TLC and MALDI-TOF mass spectrometry (FIG. 14). A dominant molecular ion peak at 894.5 (m/z) provided verification of the identity of our product (DTPA-Bis(stearylamide)_{MW}=896.4 g/mol).

Incorporation of DTPA-SA into LDL was performed at a ratio of 200:1 (probe:apoB-100) and labeling efficiency was checked indirectly by a UV spectrometer (see methods). At this ratio DTPA-SALDL was obtained at a fractional recovery of approximately 80%. Thereafter, Gd³⁺ underwent chelation with the LDL probe, then the complex was mixed with a dilute solution of tropolone to eliminate any nonspecific binding of Gd³⁺ to LDL.

In Vivo MRI:

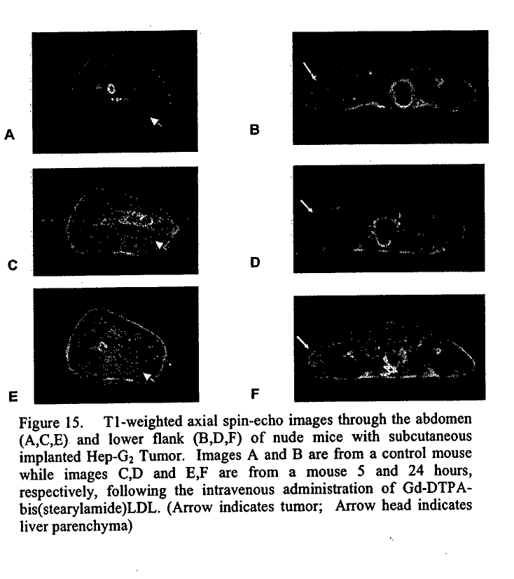

Female nude mice bearing subcutaneous HepG₂ tumors in the hind limb were used in these experiments. Gd-DTPA-SA-LDL (2.9 mM, 300 pL) was administered via the tail vein to test subjects. MRI examinations were then performed 5 and 24 hrs following contrast injection. Animals not receiving any contrast agent served as baseline controls. Representative T1-weighted axial images through the abdomen and hind quarters are displayed in FIG. 15. In baseline controls, there is little intrinsic signal contrast between the liver parenchyma/dorsal thoracic muscle and tumor/leg muscle. At 5 hrs post injection there is marked signal enhancement within the liver (70% compared to baseline control). However, at this time point the tumor enhances little relative to control (<10%). By 24 hrs the signal enhancement within the liver begins to decrease, as it is now 28% compared to baseline control. In contrast the signal enhancement within the tumor shows a striking significant increase (33% and 21% over controls and 5 hrs respectively).

The results obtained from these experiments are important as it lays the frame work for high resolution imaging of such targeting nanoplatform schemes. The accumulation/amplification mechanisms of LDLR pathway allow us to surpass the threshold (˜1 mM) for intracellular MRI detection. Moreover, the amplification mechanism also applies to the folate receptor pathway. As such, we can now safely assert that folate receptor-targeted LBNP MR imaging may indeed be feasible.

### Example 7

### LDL Nanoparticles with Bound Folate

### General Procedures

Rb. Sphaeroides is purchased from Frontier Scientific, Inc., Utah. Spirulina pacifica alga is obtained from Cyanotech Corp., Hawaii. UV-visible spectra and fluorescence emission spectra is recorded on our Perkin Elmer Lambda spectrophotometer and Perkin Elmer LS50B spectrofluorimeter, respectively. Analytical ¹H and ¹³C NMR spectra is recorded on Bruker AMX360 and 500 spectrometers available in the Department of Chemistry. Mass spectrometric data is obtained on a Micromass LC Platform using the ESI technique available in the Department of Chemistry, as well as on a matrix-assisted laser desorption ionization mass spectrometry available in the Department of Biochemistry and Biophysics.

Extraction of bacteriochlorophyll a (BChI) from Rb. Sphaeroides. Rhodobacter sphaeroides biomass (200 mL) is suspended in 1-propanol (1.5 L) and stirred at room temperature, in the dark, with constant argon bubbling for 12 h. The blue-green extract is filtered and aq. 0.5 N HCl (50 mL) is added to the filtrate. The reaction mixture is diluted with aqueous 5% NaCl (1.5 L) and extracted with dichloromethane. The combined extracts is washed with water and evaporated to dryness. The crude bacteriopheophytine is dissolved in aq. 80% TFA (200 mL) and stirred in the dark at room temperature for 2 h. The solution will then be diluted with ice water, treated with diazomethane and evaporated to dryness. The crude residue is chromatographed on silica to give pure bacteriopheophorbide (BPhe).

Culture of KB Tumor Cells. A human nasopharyngeal epidermoid carcinoma cell line, KB, is purchased from American Type Tissue Collection (ATCC, Manassas, Va.). This cell line has been selected because of putative folate receptor overexpression. KB cells is grown continuously as a monolayer at 37° C., under 5% CO₂ in folic acid deficient RPMI 1640 medium. This medium is supplemented with penicillin (100 units/mL), streptomycin (100 μg/mL), and 10% heat-inactivated fetal calf bovine serum (FBS), yielding a final folic acid concentration approximately equivalent to that in normal human serum (2-20 μg/L) (Berger, P. B. et al. Increase in total plasma homocyusteine concentration after cardiac transplantation. Mayo Clinic Proceedings 70, 125-131 (1995)).

Isolation and Purification of LDL. Human LDL and its various sub-fractions, obtained from fresh plasma of healthy, normolipidemic human donors and purified by sequential density gradient ultracentrifugation as recently reported by Lund-Katz et al. (Lund-Katz, S., Laplaud, P. M., Phillips, M. C. & Chapman, M. J. Apolipoprotein B-100 conformation and particle surface charge in human LDL subspecies: implication for LDL receptor interaction. Biochemistry. 1998 Sep. 15; 37(37):12867-74) is purchased from the Lipoprotein Core Laboratory of Dr. Lund-Katz at the Children's Hospital of Philadelphia. Briefly, blood samples is centrifuged to separate cells from plasma. The plasma density is brought to 1.063 with KBr. The plasma is centrifuged at 40,000 rpm (105,400 g) in a fixed-angle rotor (Beckman type 40) at 16° C. for 18 h, and the fractions with densities 1.009-1.063 g/ml containing LDL is separated from the upper VLDL and lower HDL and plasma protein fractions. The total LDL fraction is dialyzed overnight against two changes of the appropriate buffer.

Procedures

Synthesize LBNPs with different folate to LDL ratios and folic acid conjugated to the receptor binding Lys residues on the apoB-100 surface and optimize the chemical reaction conditions for LDL surface modification (conjugation and capping).

Conjugation Method 1: Synthesis of γ-Isomer of the Folate-LDL Conjugates

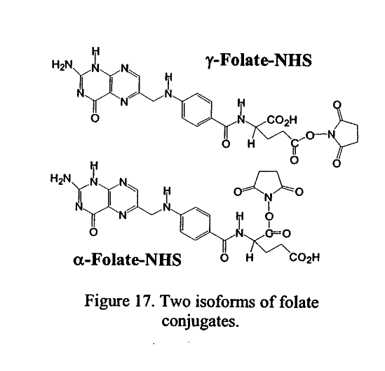

i. Synthesis and Purification of γ-NHS-folate: NHS-folate is synthesized according to the method of Lee and Low. Folic acid (5 g, 11.3 mmol; Sigma) is dissolved in DMSO (100 mL) and triethylamine (2.5 mL) and reacted with N-hydroxysuccinimide (2.6 g, 22.6 mmol) and dicyclohexylcarbodiimide (4.7 g, 22.7 mmol) overnight at room temperature. The solution will then be filtered, concentrated under reduced pressure at 37° C., and NHS-folate precipitated in diethyl ether. After washing three times in anhydrous ether and drying under vacuum, the desired NHS-folate is obtained and stored as a powder at −20° C. The product is characterized by mass spectrometry and ¹H and ¹³C NMR analysis. It is expected that NHS-folate will have two isoforms, the N-hydroxysuccinimide on the 7-carboxyl and a-carboxyl groups of folic acid (FIG. 17). These two isomers are separated by RPHPLC. Only the y-carboxyl form of NHS-folate is used for LDL conjugation.

ii. Synthesis of folate-LDL: Folate-LDL is prepared by incubating LDL (0.5 mg/mL in Tricine buffer, pH 8.5) with NHS-folate (molar ratio of NHS-folate:LDL ranging from 5:1 to 250:1) stirring at 4° C. for 48 h. The solution is spun on a low-speed centrifugation at 4° C. to remove precipitates from degraded LDL, and additional one or two centrifugation cycles may be needed to further clarify the solution. The resulting folate-LDL conjugate is dialyzed overnight at 4° C. against Tris-buffered saline (pH 7.5). Over the course of the dialysis, it is expected that unreacted NHS-folate will precipitate from solution and will subsequently be dialized and removed by membrane filtration (0.22 p.m). The resulting folate-LDL is stored for up to two weeks at 4° C. under argon for further chemical modifications. For long term storage, the folate-LDL is cyropreserved with sucrose to maintain normal physical and biological properties of LDL according to a procedure described by Masquelier et al. (Masquelier, M., Vitols, S. & Peterson, C. Low-density lipoprotein as a carrier of antitumoral drugs: in vivo fate of drug-human low-density lipoprotein complexes in mice. Cancer Research. 1986 August; 46(8):3842-7).

iii. Characterization of folate-LDL. The protein content is determined using the Lowry's method⁶². The number of folate moieties per LDL particle is calculated using spectroscopic analysis (ε₃₆₅=9120.1 M⁻¹×cm⁻¹)²⁷. By using different molar ratio of NHS-folate:LDL (5:1 to 250:1), we expect that a series of folate-LDL conjugates with the molar incorporation ratio of folate:LDL from 1:1 to 50:1 is obtained.

Protocol 1b: Capping Folate-LDL Conjugates

Rationale: Because of Lys residues' role in LDLR binding, the process of Lys modification on the apoB-100 surface of LDL is designated as “capping”. It has been shown that when about 20% of the Lys are capped, binding to the LDLR is essentially abolished. The ability of LDL to bind to the LDLR is reduced by 50% when about 8% of the Lys residues are capped. Thus, if the molar ratio of folate:LDL is smaller than 20:1, it is possible that LDLR and folate receptor is competing against each other for LDL binding and internalization. When this happens, it is necessary to cap Lys residues to block the binding of folate-LDL to LDLR. The effective Lys capping method reported so far include reaction of LDL with organic acid anhydrides (acetylation or maleylation) (Brown, M. S. et al., Journal of Supramolecular Structure. 1980; 13(1):67-81) and reaction with aldehydes (Bijsterbosch, M. K. et al., Advanced Drug Delivery Reviews 5, 231-251 (1990)) such as by treatment with malondialdehyde. Reductive methylation with formaldehyde and sodium cyanoboronhydride is also an effective Lys capping technique (Lund-Katz, S. et al., Journal of Biological Chemistry. 1988 Sep. 25; 263(27):13831-8). However, capping apoB-100 described above can often direct modified LDL particles to non-lipoprotein receptors'”. For example, acetylation of LDL induces rapid uptake by scavenger receptors on endothelial liver cells (Bijsterbosch, M. K. et al., Advanced Drug Delivery Reviews 5, 231-251 (1990)).

Protocol (Acetylation Method): The acetylation capping method is described in following steps: 1) Folate-LDL conjugate is first dialyzed in 12,000 MWCO dialysis tubing against 6 L of 0.15M NaCl at 4° C., overnight, with stirring. 2) It is sterilize by filtration using 0.45 μm Millipore filter. 3) Equal volumes of LDL and saturated sodium acetate is slowly stirred in a 100 mL beaker placed in an ice bath. 4) Every 15 min, aliquots of acetic anhydride (10 μL) is added until all of the calculated volume (50 to 200 μL depending on the degree of capping desired) has been delivered. 5) After final acetic anhydride addition, the solution is stirred on ice for at least 30 more minutes to ensure that there is no undissolved acetic anhydride. 6) The resulting acLDL is dialyzed against 3 changes of 6 L of 0.15 M NaCl, 0.3 mM EDTA, pH 7.4 at 4° C. for 48 h to remove excess acetate. 7) The product is then dialyzed against 1 or 2 changes of 6 L of 0.15 M NaCl to remove EDTA. 8) The purified acLDL is concentrated and sterilized. 9) The efficiency of the capping is verified by demonstrating increased mobility on agarose gel electrophoresis of acLDL vs sample of unmodified LDL.

A series of folate-LDL conjugates is synthesized with the folate to LDL ratios ranging from 1:1 to 50:1. Whenever necessary, capping is applied to at least 20% of Lys residues on the apoB-100 surface of LDL to ensure elimination of competing LDLR binding affinity in the resulting folate-LDL conjugates. No significant decomposition of LDL during the conjugation and capping process is expected.

Demonstrate the folate receptor binding affinity of these LBNPs by confocal microscopy by using folate receptor-overexpressing KB cells.

Protocol 2: Characterization of Cellular Uptake of Folate-LDL Conjugates in KB cells.

i. Preparation of fluorescent folate-LDL conjugates. Pyro-CE is incorporated into folate-LDL conjugates via the LDL reconstitution approach (Zheng, G. et al., Bioconjugate Chemistry. 13, 392-396 (2002)) to allow the internalization of folate-LDL in tumor cells via folate receptor to be visualized by fluorescence imaging. Thus, following our previously described procedure (Zheng, G. et al., Bioconjugate Chemistry. 13, 392-396 (2002)), 1.9 mg of dialyzed LDL is lyophilized with 25 mg starch in Siliclad-treated glass tubes. The solidified LDL is extracted three times with 5 ml of heptane at −10° C. Following aspiration of the last heptane extract, 6 mg of Pyro-CE is added in 200 pl of benzene. After 90 min at 4° C., benzene and any residual heptane is removed under a stream of N₂ in an ice salt bath. After about 45-60 min, completely dried r-LDL is solubilized in 10 mM Tricine, pH 8.2, at 4° C. for 24 hr. Starch is removed from the solution by low-speed centrifugation. The resulting Pyro-CE reconstituted folate-LDL particle [r(Pyro-CE)-FA-LDL] is used to for the following assays.

ii. Standardization: First, standard solutions of Pyro-CE is prepared in isopropanol at a concentration range of 0-2.5 pM (0-1500 ng/ml). Fluorescence measurements is performed using a Perkin-Elmer LS-50B spectrofluorometer with excitation and emission wavelengths set at 665 and 720 nm, respectively. Standard solutions of r(Pyro-CE)-FA-LDL is prepared in saline and the same volume of chloroform is used to extract the modified LDL to allow quantitation of Pyro fluorescence, since both isopropanol and chloroform are known to give a linear correlation between the Pyro concentration and the fluorescence intensity within the same range. Thus, the specific activity of r(Pyro-CE)-FA-LDL is calculated as the amount of Pyro (ng) incorporated into 1 pg of LDL protein.

iii. Visualization of cellular internalization of r(Pyro-CE)-FA-LDL and determination of its subcellular localization. About 0.5×10⁶ KB cells per well is seeded in 4-well Lab-Tek chamber slides (Naperville, Ill.). An hour before initiating an experiment, the cells is washed four times with folic acid deficient RPMI 1640 medium, then 1 mL of folic acid deficient medium is put in each well. The cells will then be incubated with the folate-LDL conjugates with or without over excess of free folic acid under these conditions. For subcellular localization studies, cells is co-incubated with LysoTracker or MitoTracker (Molecular Probes, Inc., Eugene, Oreg.) as well as with the folate-LDL conjugates. After extensive washing with phosphate-buffered saline (PBS), cells is fixed with 2% formaldehyde in PBS and Confocal microscopy is performed with a Leica TCS SPII laser scanning confocal microscope (Heidelberg, Germany). We are expecting to see the extensive fluorescence in the KB cell while the folate receptor mediated binding is indicated by the lack of fluorescence in the presence of free folic acid. Cytoplasm localization of r(Pyro-CE)-FA-LDL is identified by unmatched fluorescence with LysoTracker or MitoTracker.

iv. Quantitation of folate receptor affinity in KB cells by Scatchard analysis. KB cells is cultured in 24-well plates and allowed to grow to about 60% confluence. One day before the experiment, cells is transferred to RPMI 1640 containing 0.8% BSA instead of FBS. On the day of the experiment, cells is incubated at 4° C. for 3 hours with a series of concentrations of r(Pyro-CE)-FA-LDL with or without an excess of free folic acid. Following incubation, the culture plates is placed on ice. The cells is washed extensively with PBS. Then 1 mL of isopropanol is added to each well, and plates is gently shaken on an orbital shaker (Action Scientific, Forest Hill, Md., USA) for 15 min. The isopropanol extract of Pyro will then be transferred to a 10×75 mm glass tube, centrifuged at 3000 rpm for 15 min and the Pyro fluorescence signal is determined using the spectrofluorometer. Cells is dissolved in 0.1N NaOH for protein determination following Lowry's method. Concave-upward curvilinear Scatchard plots is produced with Prism software (GraphPad Software, Inc., San Diego, Calif.). We expect to see significant uptake of fluorescent-folate by KB cells.

Data analysis and expression of the results: Since receptors are not internalized at 4° C., only the binding of the ligand to cell surface receptors is measured. The specific binding is calculated by subtracting the nonspecific binding of r(Pyro-CE)-FA-LDL, which is determined in the presence of an excess of free folic acid, from the total binding. The ordinate of the Scatchard plots (bound/free) represents the amount of specifically bound ligand (μg of protein/mg cellular protein) divided by the concentration of unbound ligand in the reaction mixture (μg of protein/ml). The maximum binding capacity (Bₘₐₓ) is obtained from the x-axis intercept, and the equilibrium dissociation constants (K_{D}) is calculated from the slopes. K_{D} is the ligand concentration needed to bind to 50% receptors.

v. Validation of tumor models using radiolabeling assay. Since folate receptor expression level in KB cells has already been reported by using ³H-folate, this radiolabel assay is used as the gold standard for the fluorescence-based assay (protocol I). Thus, KB (10⁶) cells grown in 12-well plates is incubated at 37° C. for different times (1, 10, 30, 60, 120 min) with 50 nM ³H-folate (specific activity 34.5 Ci/mmol, American Radiolabeled Chemicals Inc., St. Louis, Mo.). At the end of incubation, cells is harvested using 0.1% Triton X-100, and the radioactivity (pmol/10⁶ cells) is determined using a scintillation counter.

Cell toxicity assay. Toxicity of r(Pyro-CE)-FA-LDL to KB cells is tested using the 3-[4,5-dimeth₆ylthiazol-2-yl]-2,5-diphenyl tetrazolium bromide (MU) method (Research Organics, Cleveland, Ohio, USA) (Mosmann, T., J. Immunol. Methods 65, 55 (1983)). Briefly, KB cells is cultured in 96-well plates with r(Pyro-CE)-FA-LDL for 72 hours. MTT is added to each well and incubated for 4 hours. Isopropanol with 0.1N HCl will then be added. Absorbance in each well is measured with a model 450 Microplate Reader (Bio-Rad, Hercules, Calif., USA). The 50% inhibitory concentration (IC50) is calculated by regression equation obtained from the dose-response absorbance curve. All experiments are repeated three times.

To prove the targeting specificity of folate-LDL conjugate to folate receptor, the affinity of folate-LDL conjugates to the folate receptors and their lack of affinity to the LDLR and scavenger receptor is both directly evaluated and also examined by a competitive binding assay using free folic acid as the receptor ligand. It is important to first characterize the folate receptor expression of KB cells (folate receptor positive) and all three control cell lines proposed for this study that includes HT1080 cells (folate receptor negative) (Moon, W. K. et al., Bioconjugate Chemistry. 2003 May-June; 14(3):539-45), (HepG₂ cells (LDLR positive) (Li, H. et al. Optical Imaging of Tumors Using Carbocyanine Labeled LDL. Acad. Rad. 11, 669-677 (2004)) and macrophages (Brown, M. S., Basu, S. K., Falck, J. R., Ho, Y. K. & Goldstein, J. L. The scavenger cell pathway for lipoprotein degradation: specificity of the binding site that mediates the uptake of negatively-charged LDL by macrophages. Journal of Supramolecular Structure. 1980; 13(1):67-81) (LDLR negative and scavenger receptor positive). For an example, if folate-LDL conjugates showed some affinity to HepG₂ cells, there are two possibilities of where such affinity comes from: 1) the LDLR binding due to the incomplete apoB-100 capping process; 2) HepG₂ cells express a certain degree of folate receptor in addition to its overexpression of LDLR. Similarly, because capping such as acetylation can lead to significant scavenger receptor binding, it is important to study the interaction between the folate-LDL conjugates and the scavenger receptor using macrophages. Furthermore, studies will also be performed to ensure that the covalent conjugation of LDL particles to the γ-carboxyl of folic acid does not compromise the latter's high affinity for folate receptor. This can be achieved by using free folic acid in a competition assay. Traditionally, radiolabeled ligand such as tritium labeled folic acids were typically used in these studies. However, since we have demonstrated the successful quantitative analysis of LDLR expression in HepG₂ tumor cells using a DiI-labeled LDL fluorescent probe; Pyro-CE probes is incorporated into the lipid core of the folate-LDL conjugates. This should allow the quantitative characterization of the folate receptor expression in KB and all control cell lines and allow the internalization process of folate-LDL by tumor cells to be visualized with confocal microscopy. Since incorporating optical probes into LDL core is an integrate part of our proposed LBNP, this should also speed up the process to implement the LBNP concept.

As compared to conventional antibodies or ligands, the use of nanoparticles allows for the multivalent attachment of molecules to the surface of the nanoparticle, which can greatly increase its binding affinity to the targeted cells (perhaps by establishing simultaneous multiple interactions between the cell surface receptor and its ligands). For example, Manchester et al. (Manchester, M. in Nanotechnology: Visualizing and Targeting CancerLa Jolla, Calif.; 2004) recently reported in the 2004 NCI Nanotechnology Workshop that by attaching multiple copies of neuropeptide Y to a Cowpea Mosaic Virus-based nanoparticle, one hundred fold binding affinity to neroblastoma tumor cells was observed compared to that of the single peptide, which is clearly due to the multivalency effect. However, it is known that some functional groups such as the free carboxyl group of the folate conjugates tend to aggregate; and aggregation of nanostructures can inactive or even precipitate the nanoparticles. In the case of folateconjugated dendrimers-based nanodevices, it was found that 2 to 3 molecules per dendrimers produced remarkable capability to target and internalize nanodevices in cells expressing the folate receptor. However, higher folate to dendrimers molar ratio actually led to the reduction of binding affinity. Thus, finding the optimal folate:LDL molar ratio is of great importance.

i. Scatchard analysis of folate receptors in different tumor models: KB, HT1080, HepG₂ and macrophage (10⁶ cells) is cultured in 24-well plates. Experiments are carried out following the protocol 2 of the R21 phase. Concave-upward curvilinear Scatchard plots are resolved using Prism software (GraphPad Software, Inc., San Diego, Calif.). We expect to see significant uptake of fluorescent LBNP by KB cells, but essentially no uptake by HT1080 cells, HepG₂ cells and macrophages.

ii. Validation of tumor models using radiolabeling assay: Since folate receptor expression levels in KB and HT1080 cell lines have already been reported by using ³H-folate, this radiolabel assay is used as the gold standard for the fluorescence-based assay (see protocol 2 of the R21 phase).

iii. Statistic Analysis: Descriptive values is expressed as mean ±SD. Statistical analyses included ANOVA for the comparison of Bₘₐₓ and K_{D} values in four different cell lines. Differences with P<0.05 is considered significant.

iv. Targeting specificity assay. To confirm the folate receptor target specificity of LBNPs, folate receptor negative HT1080 cells, LDLR positive HepG₂ cells and scavenger receptor positive (LDLr negative) macrophages is used against the folate receptor positive KB cells to test the binding affinity folate-LDL to LDLR and scavenger receptor. We will use the same assay that described for KB cells (see protocol 2 of the R21 phase).

V. Determine the optimal folate to LDL ratio for the binding of LBNP to folate receptor. As described in the R21 phase milestones, four folate-LDL conjugates with the folate to LDL ratios ranging from 1:1 to 50:1 is synthesized. These conjugates with various folate-LDL molar ratios is used to determine the optimal folate to LDL molar ratio for maximizing the folate receptor binding affinity. All experiments is carried out following methods described earlier.

From these studies, the target specificity for LBNP nanoparticles is validated. These compounds have a high folate receptor-mediated uptake but have negligible LDLR binding affinity and also have minimal scavenger receptor binding affinity.

Aggregation of modified LDL particle can result from interactions between different substituted groups and can inactivate or even precipitate the desired LBNP. This may be particularly challenging for LBNP due to the unsymmetrical nature of Lys residue distribution on the surface of apoB-100 of LDL molecules. Adjusting the molar ratio of folate to LDL and using alternative capping method are just two approaches proposed in this study.

### Example 8

### Synthesis and Spectroscopic Studies of Bacteriochlorin e6 Bisoleate (BChl-BOA)

Synthesis of bacteriopheophorbide (BChl-Acid): Bacteriopheophoride a phytyl (240 mg) was dissolved in 80% H2O/TFA solution. Under argon atmosphere, the reactive Solution was stirred at 0° C. in dark for 2 hrs. The solution was diluted in 500 ml ice-water, and organic layer washed with water three times (3×100 ml). After the organic solution was dried with Na2SO4 (5 g) for 2 hrs, the solvent was removed in vacuum. The residue crude product was chromatographed on silica gel with 5% acetone in dichloromethane and 3% methanol in dichloromethane. The desired product BChl-Acid was obtained 195 mg. Uv-vis λₘₐₓ (CH₂Cl₂): 360, 530, 752 nm, Mass calcd for C35H38N4O6:610.70, found by ESI-MS: 610. (M+), 633. (M++Na), 1243.3 (2M++Na). 1H NMR (CDCl₃, δ ppm) 8.90 (s, 1H, 5-H) 8.46 (s, 1H, 10-H), 8.44 (s, 1H, 20-H), 4.31, 4.27 (each, m, 1H, 7, 8-H), 4.11 (m, 2H, 17, 18-H), 3.88 (s, 3H, 12-CH₃), 3.48 (s, 3H, 2-CH3), 3.41 (s, 3H, 32-CH3), 3.35 (s, 2H, 81-CH2), 3.16 (s, 3H, 133-COOCH3), 2.53-2.04 (m, 5H, 171, 172 and 131-H), 1.79 (d, 3H, J=8.0 Hz, 7-CH3), 1.74 (d, 3H, J=8.0 Hz, 18-CH3), 1.11 (t, 3H, 82-CH3), 0.49 (s, 2H, center NH).

Synthesis of bisBoc-containing bacteriochlorin e6 (BChl-2BOC): The BChl-Acid 195 mg (0.319 mmol) was dissolved in 20 ml dichloromethane, and then the 4-N,N-dimethylaminopyridine 85.93 mg (DMAP, 0.703 mmol) and N-Boc-1,3-diaminopropane 167 mg (0.959 mmol) were added in the solution, respectively. After the mixture was stirred under argon atmosphere at R.T. for 30 min, the dicyclohexylcarbodiimide 131 mg (DCC, 0.639 mmol) was added the mixture solution. The mixture was continued to stir for 12 hrs. The solvent was removed by vacuum. The crude product was purified by silica gel column chromatography with 10% acetone in dichloromethane, and then with 5% methane in dichloromethane. The desired product BChl-2BOC and a side-product BChl-BOC-DCC was gotten 158 mg and 120 mg in 52.6% (0.168 mmol) yield and 37.9% yield (0.121 mmol), respectively. BChl-2Boc: Uv-vis λₘₐₓ (CH2Cl2): 354, 518, 748 nm. Mess calcd for C51H72N₈O9 941.17, found by ESI-MS: 941.1 (M+) and 964.9 (M++Na). 1H-NMR (CDCl3): 9.28 (s, 1H, 5-H), 8.66 (s, 1H, 10-H), 8.56 (s, 1H, 20-H), 6.98 (brs, 1H, 131-CONH) 5.63, 5.10 and 4.74 (each, brs, 1H, NH) 5.26 (2d, 2H, 151-CH2), 4.34, 4.24 and 4.14 (m, 4H, 7, 8, 17, and 18-H), 3.77 (m, 2H, NCH2) 3.72 (s, 3H, 152-CO2CH3), 3.69 (m, 2H, NCH2), 3.39 (s, 3H, 12-CH3), 3.38 (m, 2H), 3.32 (s, 3H, 2-CH3), 3.17 (s, 3H, 32-CH3), 2.84-2.59 (m, 4H, 2 □CH2N), 2.36 (m, 1H, 171-H), 2.22 (m, 1H, 171-H), 2.04 (m, 2H, 172-CH2), 1.94 (m, 2H, 81-CH2), 1.82 (d, 3H, 18-CH3), 1.63 (m, 5H, 7-CH3, —CH2), 1.40-1.23 (brs, 18H, —CH2, N—CO2Boc), 1.08 (t, 3H, 82-CH3), −1.22 and −1.26 (each, s, 2H, center NH).

Side product: Uv-vis λₘₐₓ (CH2Cl2): 355 nm, 517 nm, 750 nm. Mass calcd for C56H78N8O8 991.27, found by ESI-MS: 992.23 (M++1) and 1015.24. (M++1+Na).

Synthesis of diamino bacteriochlorin e6 (BChl-2NH2) and bacteriochlorin e6 bisoleate (BChl-BOA) (Scheme 4): The BChl-2BOC 40 mg (0.432 mmol) was dissolved in 1.5 ml TAF. The solution was stirred first in ice-water bath under argon atmosphere for 1 hrs, and then at RT for 1 hrs. After the TFA was removed in vacuum, the crude product BChl-NH2 did not need to be purified further, and used directly for the next reaction. Mass calcd for C41H56N8O5 740.93, found by ESI-MS 741.1 (M+).

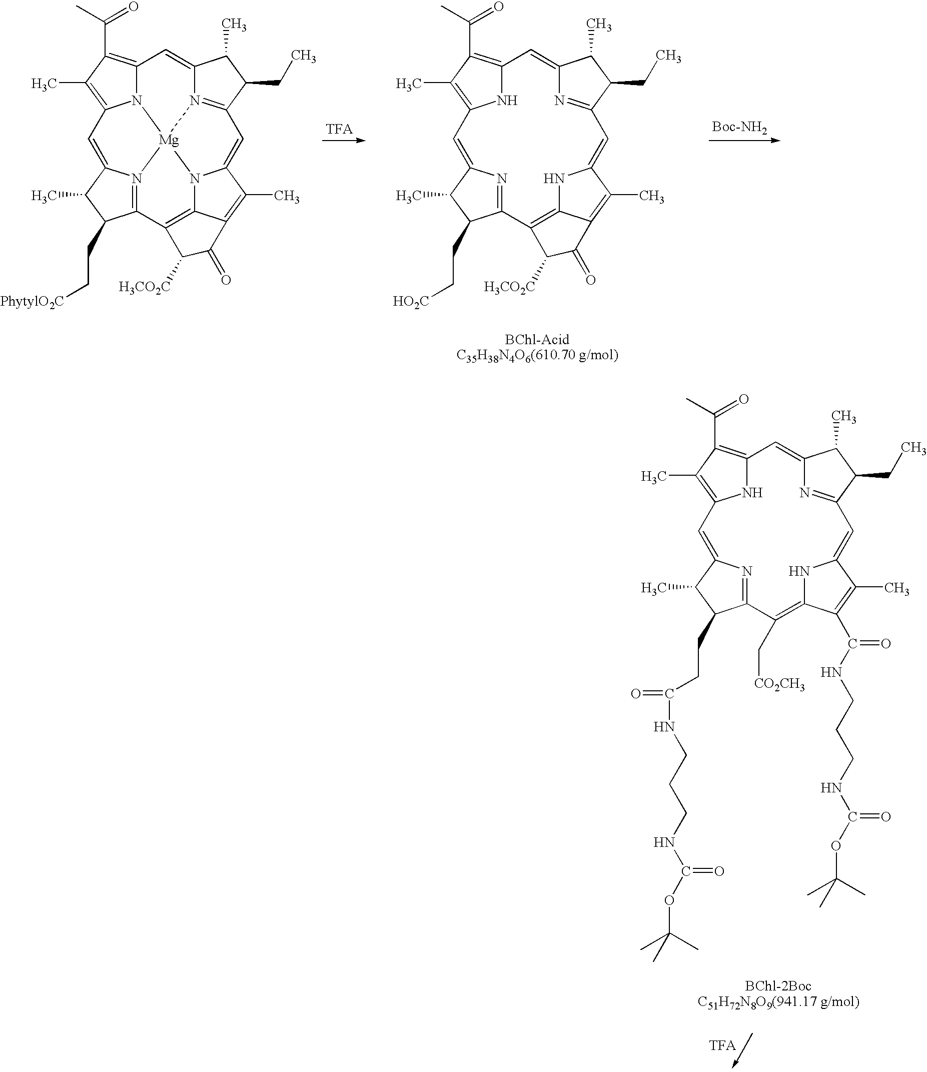

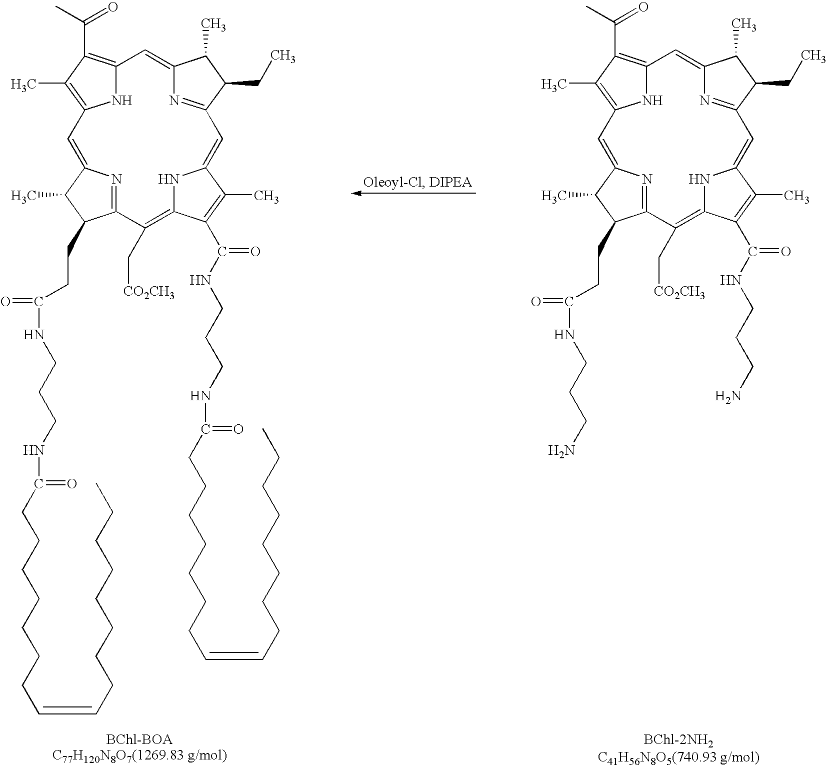

The crude product BChI-NH₂ was dissolved in 5 ml dichloromethane, and then 0.15 ml diisopropylethylamine (DIPEA) was added the solution. After the mixture was stirred under argon atmosphere at RT for 0.5 hrs, oleoyl chloride (ole-Cl) 0.15 ml was added into the mixture solution, and continued to e stirred for 4 hrs at RT. The solution was poured into 50 ml ice water and washed with ice water three times 3×20 ml. The organic layer was dried over anhydrous NaSO410 mg for 1 hrs. After the solvent was removed in vacuum, the crude product was purified by silica get column chromatography with 5% methanol in dichloromethane. The desired product was obtained in 69 mg. Uv-vis λₘₐₓ (CH2Cl2): 355, 516, 747 nm Mass. calcd for C77H120N8O7 1269.83, found by ESI-MS 1270.1 (M+) and 1292.8 (M++Na). 1HNMR (CDCl3, δ ppm): 9.28 (s, 1H, 5-H), 8.66 (s, 1H, 10-H), 8.57 (s, 1H, 20-H), 7.16, 6.36, 5.98 and 5.82 (each, brs, 1H, NH), 5.31-5.15 (m, 6H, 151-CH2 and vinyl of oleoyl), 4.33-4.13 (m, 4H, 7, 8, 17 and 18-H), 3.73 (m, 2H, NCH2), 3.68 (s, 3H, 152-CO2CH3), 3.56 (s, 3H, 2-CH3), 3.47 (m, 2H, NCH2), 3.31 (s, 3H, 12-CH3), 3.16 (s, 3H, 32-CH3), 2.91-2.83 (m, 4H, 2□ CH2N), 2.35 (m, 1H, 171-H), 2.05-2.01 (m, 5H, 171-H, —CH2), 1.98-1.86 (m, 16H, —CH2) 1.82 (d, 3H, 18-CH3), 1.76 (brs, 4H, CH2), 1.61 (d, 3H, 7-CH3), 1.55 (m, 4H, —CH2), 1.45 (m, 2H, —CH2), 1.23-1.17 (brs, 34H, other H in oleoyl), 1.08 (t, 3H, 82-CH3), 0.85 (m, 6H, —CH3), −1.24 and −1.28 (each s, 1H, center NH).

Synthesis of palladium (II) complex of BChl-BOA (Pd—BChl-BOA): To maximize the photostability of BChl-BOA dye to improve LDL reconstitution efficiency, palladium (Pd) was inserted into the center of the BChl ring following a procedure described below: 6-O-palmitoyl-L-ascorbic acid (30 mg, 72□mol) was dissolved in MeOH (10 mL) and argon was passed through the solution. BChl-BOA (23 mg, 18 □mol) and Pd (II) diacetate (10 mg, 45 □mol) was dissolved in CHCl3 (10 mL, degassed) and added to the methanolic solution. The mixture was kept under inert atmosphere by stirring and the reaction progress was monitored by recording the absorption spectra of small reaction portions every 15 min. After about 60 min, the reaction was completed and solvent were evaporated. The crude was chromatographed on silica plate with 5% MeOH/CH2Cl2 to afford the desired product in 60% yield. The structure of Pd—BChl-BOA was confirmed by NMR, mass (see FIG. 8). Comparing their UV-visible spectra (see FIG. 9), the Qy band of the Pd—BChl-BOA is at the same wavelength (˜750 nm) as that of metal free BChl-BOA but has much sharper peak shape, whereas the single Soret band in BChl-BOA (357 nm) is split into two distinctive bands at 332 nm and 388 nm, a characteristic of Pd—BChl complex.

### Example 9

### MRI-Based LBNP Contrast Agents

Rationale. High-resolution contrast enhanced MRI is one of the most useful techniques for screening tumors and other anatomical abnormalities. Because of sensitivity limitation of the current MRI techniques, efficient recognition requires a very high capacity target like fibrin, which is present in sufficient quantity to be seen with simple targeted Gd chelates, or targets accessible to the blood stream that can be bound with a Gd cluster, polymer or an iron particle. This is possible presently only in a limited target set. For example, the seminal work by Sipkins et al. (Sipkins, D. A. et al. ICAM-1 expression in autoimmune encephalitis visualized using magnetic resonance imaging. J Neuroimmunol 104, 1-9 (2000)) demonstrated that paramagnetic immunoliposomes targeted to the integrin receptor, intercellular adhesion molecule-1 (ICAM-1), could be used to visualize altered ICAM-1 expression in autoimmune encephalitis using MRI. More recently Lanza and Wickline et al. (Anderson, S. A. et al., Magnetic Resonance in Medicine. 2000 September; 44(3):433-9) developed a fibrin-targeted paramagnetic nanoparticle contrast agent for high-resolution MRI characterization of human thrombus. In their approach, the contrast agent is a lipid-encapsulated perfluorocarbon nanoparticle with numerous Gd-DTPA complexes incorporated into the outer surface. The nanoparticles themselves provide little or no blood-pool contrast when administrated in vivo, but when they bind and collect at a targeted site, such as a thrombus, they modify the T1-contrast of the tissue substantially. Thus, they inherently yield high signal-to-noise ratios. However, unlike imaging fibrin, an extra-cellular target, intracellular MRI imaging is particularly challenging because the minimum concentration of MRI agents required for the MRI detection limit is much higher (˜1 mM) than the extracellular target (40 μM). In spite of this difficulty several attempts have been made to visualize intracellular contrast agents, Wiener et al. (Wiener, E. C. et al., Investigative Radiology. 1997 December, 32(12):748-54) in 1995 demonstrated that with folate-conjugated DTPA-based dendrimers they could observe receptor mediated uptake of their agent by tumor cells which overexpressed the folate receptor and achieved 17% MRI contrast enhancement at 24 h following its administration. Alternatively, Weissleder et al. (Weissleder, R. et al., Nat Med 6, 351-355 (2000)) have successfully utilized iron oxide particles targeted to the transferrin receptor to visualize tumors overexpressing this receptor. In this project, folate-conjugated LBNP to deliver MRI agents to tumor cells through folate receptor.

Preparation of Gd-DTPA-LBNP with Different Gd Payloads.

Rationale: In general two classes of MRI contrast agents are delivered via LBNP to tumor cells. These are the iron oxide and lanthanide base agents. While iron oxide particles offer high sensitivity in T2-weighted images via magnetic susceptibility effects, the actual tumor to tissue contrast enhancement may be limited as tumors may already be quite hypo-intense, on a T2-weighted image. Thus detecting differences by making a dark image further darker may be problematic. Among the lanthanide base agents, the gadolinium (Gd³⁺) metal was selected due to its optimal MR properties. Although single molecules of Gd complexes induce small relaxation effects of surrounding water, we can enhance these effects several fold by incorporating a high payload of Gd-DTPA into the phospholipid monolayer shell of LDL using various lipid anchors. This will allow the Gd complex to be in an environment that is readily accessible for water interactions.

i. Preparation of Gd-DTPA-LBNP using DTPA-Bis(stearylamide) (DTPA-SA). DTPABis(stearylamide) (DTPA-SA) is prepared by methods similar, to those previously reported by Jasanada et al. (Jasanada, F. et al. Indium-111 labeling of low density lipoproteins with the DTPA-bis(stearylamide): evaluation as a potential radiopharmaceutical for tumor localization. Bioconjugate Chemistry. 1996 January-February; 7(1):72-81); briefly stearylamine (2 mmol) in chloroform (40 ml) is slowly added to DTPA (1.1 mmol) in DMF (50 ml). After 2 hours of stirring at 40° C. the solution is cooled at 4° C. for 2 hours. The white precipitate is filtered, washed with acetone and dried overnight at 80° C. The precipitate will then be crystallized in boiling ethanol (800 ml). After 24 hours at room temperature, the small crystals is collected by filtration and washed with water (800 ml, 80° C. for 3 hours) and chloroform (800 ml, reflux for 3 hours) to eliminate unreacted DTPA and SA. The purity of the product is checked with TLC and MALDI-TOF mass spectrometry.

ii. Incorporation of DTPA-SA into LDL: A 3.0 mM solution of DTPA-SA is prepared by dissolving the crystals in aqueous ammonia solution (NH₄OH/NH₄CI, pH 9, 0.15 M) with vigorous stirring. Once dissolved, the solution is diluted to a concentration of 1.5 mM. DTPA-SA and LDL is added at a molar ratio of 200:1. LDL (1 mg) is used for each reaction. Tris-buffered saline (1 mL) is added to an appropriate aliquot of DTPA-SA solution. HCl (1 M) is added drop-wise to reduce the pH of the solution to 7.5. LDL (1 mg) is then added to the solution together with additional Tris-buffered saline to bring the concentration of LDL to 0.4 mg/mL in the final solution. The mixture is allowed to stir under argon for one hour at room temperature. Thereafter the sample is filtered through a 0.22 pm membrane filter and dialyzed against Tris-buffered saline overnight (16 h) at 4° C. Over the course of the dialysis, unincorporated DTPA-SA precipitates out of solution. Membrane filtration (0.22 pm) following dialysis, removes the precipitate.

iii. Determination of DTPA-SA Incorporation: DTPA-SA concentrations can be determined indirectly by UV spectrometry. A sample of stock DTPA-SA solution is diluted 10-fold (to 150 μM) to generate standards for the assay. One-hundred microliter aliquots is further diluted to the following concentrations 15, 30, 45, 60 and 75 μM. Tris-buffered saline and solutions of zinc sulfate (60 μM) and dithizone in CHCI₃ (˜1 μM) is added to aliquots of DTPA-SA in a following manner as shown in Table 2.

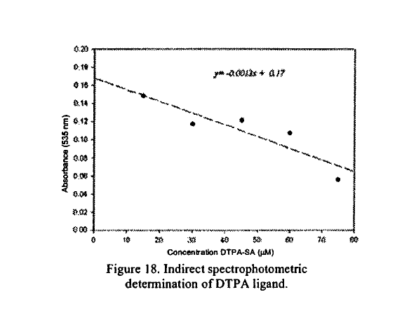

The standards, Tris-buffered saline and zinc sulfate solutions will first be mixed. An excess molar ratio of zinc to DTPA is typically utilized in this assay. The chelating DTPA-SA combines with an equivalent amount of zinc (1:1) leaving the remaining free zinc to react with subsequently added dithizone. Once the dithizone-CHCl₃ is added the sample is mixed (high vortex for 10 sec) and the organic phase removed for analysis. During this step the free zinc is extracted by the chloroform solution containing dithizone. UV spectrometry (λ=535 nm) will then be performed on the organic phase. The decrease in the absorbance of the zinc dithizonate complex is directly proportional to DTPA-SA present in the aqueous phase. FIG. 18 depicts a typical calibration curve for DTPA-SA. The calibration curve obeys Beer-Lambert's law from 0 to 60 μM. Measurements is carried out on a Perkin Elmer UVNIS spectrophotometer (Perkin-Elmer Ltd., Beaconsfield, Buckinghamshire, England).

iv. Determination of LDL labelling efficiency: Knowing that one LDL particle contains only one Apo-B protein, the molar concentration of LDL is determined with respect to Apo-B protein (molecular weight 550 kDa). A commercial Lowry protein assay kit (Sigma-Aldrich, St. Louis, Mo.) is used to measure LDL. Initial results indicate that approximately 120-160 DTPA-SA molecules are incorporated per LDL particle (60-80% labeling efficiency).

v. Gd Labelling: Gadolinium citrate solution is prepared by adding GdCl₃ in HCl (17.5 μmol) to a solution of sodium citrate (87.5 μmol). The carrier citrate is used as a transfer agent to avoid the formation of gadolinium hydroxides. The pH of the Gd-citrate solution is adjusted to 7.4. Gd labeling of LDLDTPA-SA is performed by slowly adding Gd-citrate to a solution of LDL-DTPA-SA at a metal:ligand ratio of 1:1. Following incubation for 1 hour at room temperature with gentle stirring the final product is filtered.

vi. Determination of the Gd:LBNP molar ratio. Gd content of Gd-DTPA-LBNP is analyzed by the Toxicology New Bolton Center (Kennett Square, Pa.) using inductively coupled plasma mass spectrometry (ICP-MS). Number of Gd per LDL particle is calculated based on Gd content (ICP-MS results) and protein content of the sample, as measured by Lowry assay.

### Estimation of Longitudinal Relaxivity of Gd-DTPA-LDL in Solution

Gd-DTPA-LBNP is dissolved in saline or serum at the concentration of 30, 60 and 100 μM at 37° C., and the longitudinal relaxation time (T₁) of the solution is determined on a 4.7 T spectrometer using a T₁ estimation protocol provided by the manufacturer (Varian, Palo Alto, Calif.) shown as follows: 1/T₁=1/T₁₀+α×C, where T₁₀ is the longitudinal relaxation time of saline or serum without Gd-DTPA-LBNP, α is the relaxivity (relaxation rate per mM of metal ion) and C is the concentration of Gd-DTPA (mM), which is measured accurately by the ICP/MS technique. α(s−¹mM⁻¹) is obtained by solving the above equation.

### MRI of Folate Receptor In Vivo

i. In vivo MRI Imaging: Three groups of five female nude mice with matched tumors (5-7 mm) are employed in this study. Two cohorts have KB tumors (folate receptor positive) and HT1080 tumors (folate receptor negative), respectively. The remaining cohort has both tumors planted as described in protocol 2b will serve as the control cohort. MRI is performed on 4.7 T horizontal bore [NOVA spectrometer (Varian, Palo Alto, Calif.) equipped with a 12 cm gradient set having a maximum strength of 25 gauss/cm. Mouse bearing subcutaneous KB or HT1080 tumor or both is anesthetized by 1% isoflurane air mixture and a rectal temperature probe and a tail vein catheter is placed. The position of the tumor implantation is such that the tumor and the liver are visualized on the same slice. The core temperature is monitored and maintained at 37±0.1° C. using a small animal monitoring system (SA Inc., Stony Brook, N.Y.), which controls the flow and the temperature of warm air directed to the bore of the magnet. Given that the intracellular space constitutes between 50 and 80% of the tumor space, the intracellular accumulation of targeted agents and in turn its effect on contrast enhancement within the tumor cells may be significant.

A spin echo sequence is used to acquire 5-7 slices through the tumor in the transverse plane (TR/TE=500/15 ms, matrix=256×128 and FOV=4×2 cm, slice thickness=1 mm with no interval, Signal average=4).

Precontrast longitudinal relaxation time (T₁) map of tumor is estimated by using the TOMROP sequence (also referred to as Look-Locker sequence) with TI=60 ms, number of TI interval=16, TRITE=12/8 ms, matrix=256×128 and average=2. The flip angle of read pulse is 10° and its effect on longitudinal magnetization is considered into the construction of T₁ map.

Following T₁₀ measurement, Gd-LBNP is infused into the tail vein of the animal while still in the magnet and T₁ weighted images as well as T₁ map is acquired 1 and 4 hour(s) after infusion. The animal is removed from the magnet and then scanned again at 24 hrs post infusion.

ii. Imaging Data Analysis: The specific uptake of Gd-LBNP into tumor mediated by folate receptor results in the accumulation of Gd-DTPA inside the tumor cells. Image contrast enhancement and T₁ measurements is performed to quantify the amount of contrast agent in specific tissues. Contrast enhancement values is calculated by relating the pixel intensity values, I, of the target tissue (liver or tumor) to an unaffected tissue (skeletal muscle) according to the following equation:

% Contrast Enhancement=(RIₚₒₛₜ−RIₚᵣₑ)/RIₚᵣₑ×100

where RIₚₒₛₜ is the relative intensity (Iₗᵢᵥₑᵣ or Iₜᵤₘₒᵣ/I_{muscle}) following infusion and RIₚᵣₑ is the relative intensity (Iₗᵢᵥₑᵣ/I_{muscle}) prior to infusion.

Estimation of T₁ of tumor provides a quantitative way for comparing the accumulation of Gd-DTPA at different time points or when comparing to other tissue/organ, such as brain, which also expresses folate receptor. Percent change of T₁ at various time points post-contrast from T₁ is calculated for tumor, liver and muscle.

Both of the methods (% contrast enhancement and T₁ map) for data analysis provide information about the uptake of the receptor targeted contrast agent. While measuring % contrast enhancement is a relatively simple and quick method, T₁ map, on the other hand, is quantitative but requires long scan times to generate. In addition, motion and altered gating may prove problematic over the long duration of the T₁ map scan. If such difficulties should arise we will focus primarily on the result generated from the % contrast enhancement method.

iii. Statistic Method: This protocol requires ANOVA or Repeated Measures of ANOVA analyses. These and other statistical analyses are currently performed using an in-house computer program written for our UNIX system. For many parameters, we have no prior estimate of this ratio (reciprocal of the coefficient of variation of the difference) and so we will conduct the experiment in two stages, an initial one with n=5 to estimate means and variances and a second stage with sample sizes chosen using first stage results. Because effect sizes assessed with small samples are highly variable, we will estimate a confidence interval for the effect and use a pessimistic estimate (lower end of the interval) as the basis of the sample size calculation. Additionally, we will perform a “conditional power calculation” which does not use stage I data to project effect sizes but rather performs the usual two sample Student's t-test power calculation by fixing the data collected so far and adding new hypothesized data to it.

### Protocol 3d: Preliminary Toxicity Studies of Gd-DTPA-LBNP:

For preliminary acute toxicity studies, a reported procedure for evaluating MRI contrast agent toxicity is followed (Bogdanov, A. A., Jr. et al. A new macromolecule as a contrast agent for MR angiography: preparation, properties, and animal studies. Radiology 187, 701-706 (1993)). Thus, thirty times the projected clinical dose of Gd-DTPA-SA-LDL (0.6-1 mmol/kg of body weight in 300 μL of saline solution) is injected intravenously (n=3) and/or intraperitoneally (n=3) into mice. Animals is observed for 4 weeks for clinical signs of toxicity and then killed. Alternatively, the agent is injected repeatedly in another group of mice (n=5; five to six injections in 2 weeks; total dose 0.2 mmol/kg body weight). Specimens of kidneys, liver, and spleen is processed for histological examination and is compared with tissue from normal control animals. Based on the nature of our LBNP design, we expect little or no adverse effect at this dose.

Gd-DTPA Labeled NIRF-LBNP as Combined MR/NIRF Imaging Probes.

A number of quantitative 3D tomographic NIR optical imaging techniques have been recently developed and combined with MRI to yield highly detailed anatomic and molecular information in vivo^{75, 76}. The NIR dyes we proposed to develop are reconstituted into the LBNP lipid core (end-product 1), whereas the gadolinium complexes are incorporated into the LBNP phospholipid monolayer (end-product 2). Although out of the scope of the present project, it is logical to combine these two imaging modalities using LBNP nanoparticles as the common platform in future studies. This will combine the strength of both MRI (high-resolution/anatomic) and NIRF (high sensitivity) to thus facilitate the noninvasive detection of cancers.

### Example 10

### Folate Receptor Targeted LBNP

A folate receptor-targeted low-density lipoprotein (LDL) was prepared by conjugating folic acid to lysine residues of apoB-100 protein. This turns off the LDL receptor (LDLR) binding and redirects the resulting conjugate to cancer cells via folate receptors.

Lipoproteins are a class of natural nanostructures responsible for the transport of cholesterol and other lipids in the blood circulation. They share a common structure of an apolar core surrounded by a phospholipid monolayer but differ significantly in their sizes as well as in their respective embedded apoproteins, which are recognized specifically by corresponding lipoprotein receptors. Being endogenous carriers, lipoproteins are not immunogenic and escape recognition by the reticuloendothelial system). Thus, nanoplatforms made of these proteins may provide a solution to the common biocompatibility problems associated with most synthetic nanodevices.

This example reports the concept of exploring nature lipoprotein nanoparticles as chemical building block for making diverse, multifunctional and biocompatible nanoplatforms, focusing on low-density lipoprotein (LDL) (22 nm) as a prototype. In our LBNP design (see FIG. 1), the multifunctionality is achieved by incorporating diagnostic and/or therapeutic agents into the LDL core and on its surface monolayer. The diverse targeting of LBNP is achieved by conjugating different tumor-homing molecules to the Lys residues exposed on the apoB-100 surface of LDL. This turns off the LDLR binding and redirects the resulting LBNP nanoparticles to cancer cells via non-LDLR cancer signatures (e.g, Her2/neu, αᵥβ₃ integrin).

Folic acid (FA) was chosen as the tumor homing molecule. It has high affinity (Kd≦10⁻⁹M) to folate receptors (FR), which are overexpressed in epithelial colorectal cancers. Moreover, it has been shown that when FA is covalently linked to a macromolecules (<60 nm) via its γ-carboxyl moiety, it retains high affinity to FR. To functionalize LBNP, tetra-t-methyl-silicon phthalocyanine bisoleate (SiPc-BOA) and 1,1-dioctadecyl-3,3,3,3-tetramethylindocarbocyanine perchlorate (DiI) were used. SiPc-BOA is an analog of Pc4, a well-known PDT agent. Because its central silicon atom allows axial coordination of two oleate moieties, SiPc-BOA is nonaggregatable and highly lipophilic, which proved to be essential to achieve high payload via LDL reconstitution. DiI is a lipid-anchored, carbocyanine-based optical probe known to intercalate into LDL phospholipid monolayer. Thus, by using SiPc-BOA and DiI together with FA-conjugated LDL (LDL-FA), this study demonstrates that LDL nanoparticles carrying diagnostic/therapeutic agents are delivered to a disease target other than LDLR.

To synthesize FR-targeted LBNP, LDL was first functionalized with DiI (surface loading) or SiPc-BOA (core loading), followed by FA conjugation for redirection. To label LDL surface with DiI, LDL was incubated with 60-fold molar excess of DiI according to a literature procedure. The resulting complex, DiI-LDL, has a molar ratio of 55:1. To form the SiPc-BOA reconstituted LDL (r-Pc-LDL), we have recently achieved a very high SiPc-BOA payload (3000:1) using a modified Kreiger method. Unlike the cargo loading described above, the attachment of a folic acid or any other tumor homing molecule to the apoB-100 of a LDL is unprecedented. Thus, it began with increasing pH from 7.4 to 9.4 by dialyzing LDL against a Nal-I₂PO₄/H₃BO₃ buffer. This was followed by reacting LDL with 250-fold molar excess of FAN-hydroxysuccinimide ester at 4° C. for 30 h. Upon completion, the mixture was centrifuged at low speed to remove any degraded LDL. In the final step, the crude LDL-FA was dialyzed against EDTA buffer to adjust pH back to 7.4 and to remove any unreacted FA. This yielded two pure complexes, DiI-LDL-FA and r-Pc-LDL-FA. Electronic microscopy studies revealed only a slight increase in mean particle size (26.12 f 3.00 nm for the former and 24.01±4.30 nm for the latter) over native LDL (20.00±2.70 nm). To determine how many FA molecules were attached to each LDL nanoparticle, standard curves (linear correlation between absorbance and concentration) of FA and LDL were generated at 280 nm. Based on the LDL standard curve, LDL absorbance was derived from LDL concentration which was determined by Lowry's method. FA absorbance was then calculated using formula A_{FA}⁼A_{LDL-FA}−A_{LDL} where A is absorbance. Based on the FA standard curve, FA concentration was derived and FA payload was calculated. Thus, the molar ratios of two nanoparticles, DiI-LDL-FA and r-Pc-LDLFA, were determined to be 50:1:170 and 3000:1:170 respectively.

To confirm that the LDL redirection strategy works, confocal microscopy and flow cytometry studies were performed on DiI-LDL-FA and r-Pc-LDL-FA using following cell lines: 1) Human nasopharyngeal epidermoid carcinoma, KB cells (FR overexpression, FR^{+′}),² 2) Chinese hamster ovary (CHO) cells (lack of detectable FR expression, FR⁻), 3) human fibrosarcoma, HT1080 (lack of detectable FR expression, FR⁻), and 4) human hepatoblastoma G2 (HepG₂) cells (LDLR overexpression, LDLR⁺). Thus, FR-mediated uptake of DiI-LDL-FA was first tested in KB cells (FR^{+′}). As expected, confocal images showed strong accumulation of DiI-LDL-FA throughout the whole cell except for the nucleus (FIG. 19A-2). To determine the FR-specificity of DiI-LDL-FA, three sets of control experiments were designed and following evidence were obtained: 1) Excess of free FA, the native ligand of FR, completely blocked the DiI-LDL-FA uptake in FR positive KB cells (FIG. 19A-3), whereas excess of native LDL had no effect on its uptake (FIG. 19A-4); 2) no DiI-LDL-FA uptake was observed in both FR negative CHO and HT1080 cells (FIGS. 19B-6 and 19B-8); 3) in sharp contrast to the strong accumulation of DiI-LDL in HepG₂ cells (LDLR⁺) (FIG. 19C-11), no accumulation of DiI-LDL-FA in HepG₂ cells (FIG. 19C-10) was observed indicating its diminished binding affinity to LDLR. To provide quantitative evidence of the binding specificity of DiI-LDL-FA toward FR, flow cytometry assay was performed in KB cells (FR⁺). The results showed both a concentration-dependent uptake of DiI-LDL-FA (FIG. 19D-1) and a concentration-dependent inhibition of DiI-LDL-FA uptake by free FA (FIG. 19D-2). Taking together, these data suggest DiI-LDL-FA uptake in KB cells (FR⁺) is FR-specific and FA conjugation successfully redirect LDL from LDLR to FR.

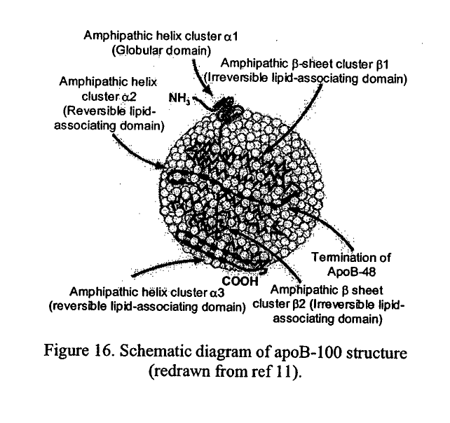

Similarly, FR-specific uptake of r-Pc-LDL-FA in KB cells was also confirmed. Briefly, r-Pc-LDL-FA emitted strong fluorescence in FR^{t} cells (KB) (FIG. 19E-12) but not in FR⁻ cells (CHO) (FIG. 19E-15). This fluorescence signal was inhibited by free FA (FIG. 19E-13) but not by native LDL (FIG. 19E-14). While r-Pc-LDL produced strong fluorescence in LDLR⁺ cells (HepG₂) (FIG. 19E-17), incorporation of multiple FA moieties led to the diminished fluorescence signal (FIG. 19E-16). These data suggest that FA conjugation successfully redirects r-Pc-LDL-FA from LDLR to FR.

### CONCLUSION

It is known that modifying the Lys residues exposed on apoB-100 surface (225 out of total 357 Lys) leads to partial or complete loss of the original LDLR binding. For example, the ability of LDL to bind to LDLR is reduced by 50% when about 8% of the Lys residues are capped via reductive methylation via sodium cyanoboronhydride, whereas 20% capping abolished the LDLR binding. The fact that ˜170 FA attached to a single LDL molecule in either DiI-LDL-FA or r-Pc-LDL-FA resulted in a 50% capping of Lys residues. This should sufficiently abolish their ability to bind to LDLR. Moreover, the redirection strategy, the center piece of the LBNP concept, is also inspired by nature. For example, acetylation of LDL induces rapid uptake by scavenger receptors on endothelial liver cells, which is one of the major LDL clearance pathways. The fact that modified LDL can be taken up by specific receptors other than LDLR indicates that it is possible to redirect the LDL targeting to various cancer signatures (e.g., FR for ovarian cancer, αᵥβ₃ integrin receptor for tumor angiogensis, and Her2/neu receptor for breast cancer) by incorporating specific tumor-homing molecules into the apoB-100 molecule. Indeed the successful redirection of functionalized LDL nanoparticles from LDLR to FR described in this communication provided the first direct evidence for our LBNP concept.

Rapid advance of nanotechnologies has generated reasonable hopes for the conversion of cancer from a fatal to a chronic disease. However, before the unique features of the promising field of nano-medicine can be fully utilized, current challenges including biocompatiblity and biodegradablity need to be addressed. LDL is a biocompatible and biodegradable nanoparticle by nature and is known for its ability to carry large diagnostic or therapeutic cargos both on its surface and inside its apolar core. However, the fact that each LDL has a single copy of apoB-100 makes its binding to LDLR monovalent and also limits its application to those LDLR-related diseases. Thus, redirecting LDL from LDLR to other cancer signatures by conjugating multiple tumor homing molecules to Lys residues of apoB-100 not only addresses the multivalency issue but also expands its current capabilities to many non LDLR-related diseases, making LDL a true nature's nanoplatform.

What is claimed is:

1. A non-naturally occurring lipoprotein nanoplatform comprising:
a. at least one lipid;
b. at least one active cell surface receptor ligand;
c. at least one apoprotein; and
d. at least one active agent;
wherein said active cell surface receptor ligand is not a low-density lipoprotein receptor ligand or a high-density lipoprotein receptor ligand and wherein said active cell surface receptor ligand is covalently bound to said apoprotein, and wherein said components (a), (b), (c) and (d) associate to form a non-naturally occurring lipoprotein nanoplatform.

2. The lipoprotein nanoplatform of claim 1, wherein said lipid is selected from the group consisting of phosphatidylcholine, lysophosphatidylcholine, phosphatidylethanolamine, phosphatidylserine and phosphatidylinositol, and combinations thereof.

3. The lipoprotein nanoplatform of claim 1, wherein said nanoplatform is from 5 to 100 nm in diameter.

4. The lipoprotein nanoplatform of claim 3, wherein said nanoplatform is from 8 to 80 nm in diameter.

5. The lipoprotein nanoplatform of claim 1, wherein said cell surface receptor ligand is a ligand for a receptor over-expressed in cancer cells.

6. The lipoprotein nanoplatform of claim 1, wherein said cell surface receptor ligand is a ligand for a receptor over-expressed in cardiovascular plaques.

7. The lipoprotein nanoplatform of claim 6, wherein said cell surface receptor ligand is a ligand for a receptor selected from the group consisting of folate, Her-2/neu, integrin, EGFR, metastin, ErbB, c-Kit, c-Met, CXR4, CCR7, endothelin-A, PPAR-delta, PDGFR A, BAG-1, and TGF beta.

8. The lipoprotein nanoplatform of claim 7, wherein said integrin receptor is αᵥβ₃.

9. The lipoprotein nanoplatform of claim 7, wherein said cell surface receptor ligand is a folate receptor ligand.

10. The lipoprotein nanoplatform of claim 1, wherein said cell surface receptor targets a specific tissue.

11. The lipoprotein nanoplatform of claim 10, wherein said tissue is lung, skin, pancreas, retina, prostate, ovary, lymph node, adrenal, liver, breast, digestive system, and renal.

12. The lipoprotein nanoplatform of claim 1, wherein said active agent is a lipophilic compound.

13. The lipoprotein nanoplatform of claim 12, wherein said lipophilic compound is selected from the group consisting of acetanilides, anilides, aminoquinolines, benzhydryl compounds, benzodiazepines, benzofurans, cannabinoids, cyclic peptides, dibenzazepines, digitalis gylcosides, ergot alkaloids, flavonoids, imidazoles, quinolines, macrolides, naphthalenes, opiates (or morphinans), oxazines, oxazoles, phenylalkylamines, piperidines, polycyclic aromatic hydrocarbons, pyrrolidines, pyrrolidinones, stilbenes, sulfonylureas, sulfones, triazoles, tropanes, and vinca alkaloids.

14. The lipoprotein nanoplatform of claim 12, wherein said lipophilic compound comprises at least one hydrocarbon chain comprising at least 10 carbons, wherein said hydrocarbon chain comprises at least one cis-double bond or branched by at least one side chain.

15. The lipoprotein nanoplatform of claim 12, wherein said lipophilic compound comprises at least one oleate moiety, cholesterol oleate moiety, cholesteryl laurate moiety or phytol moiety.

16. The lipoprotein nanoplatform of claim 1, wherein said active agent is an molecule comprising a lipophilic and a hydrophilic component.

17. The lipoprotein based nanoplatform of claim 1, wherein said active agent is a diagnostic agent or a therapeutic agent.

18. The lipoprotein based nanoplatform of claim 17, wherein said active agent is a diagnostic agent.

19. The lipoprotein based nanoplatform of claim 18, wherein said diagnostic agent is selected from the group consisting of a contrast agent, a radioactive label and a fluorescent label.

20. The lipoprotein based nanoplatform of claim 19, wherein said diagnostic agent is a contrast agent.

21. The lipoprotein based nanoplatform of claim 20, wherein said contrast agent is selected from the group consisting of an optical contrast agent, an MRI contrast agent, an ultrasound contrast agent, an X-ray contrast agent and radio-nuclides.

22. The lipoprotein based nanoplatform of claim 21, wherein said contrast agent is an optical contrast agent.

23. The lipoprotein based nanoplatform of claim 20, wherein said contrast agent is an MRI contrast agent.

24. The lipoprotein based nanoplatform of claim 23, wherein said MRI contrast agent is an iron oxide or a lanthanide base.

25. The lipoprotein based nanoplatform of claim 24, wherein said MRI contrast agent is a lanthanide base.

26. The lipoprotein based nanoplatform of claim 25, wherein said lanthanide base is gadolinium (Gd³⁺) metal.

27. The lipoprotein based nanoplatform of claim 17, wherein said active agent is a therapeutic agent.

28. The lipoprotein based nanoplatform of claim 27, wherein said therapeutic agent is an anticancer agent.

29. The lipoprotein based nanoplatform of claim 28, wherein said anticancer agent is selected from the group consisting of a chemotherapeutic agent, a photodynamic therapy agent, a boron neutron capture therapy agent or a radionuclide for radiation therapy.

30. The lipoprotein based nanoplatform of claim 29, wherein said anticancer agent is a photodyanamic therapy agent.

31. The lipoprotein based nanoplatform of claim 30, wherein said photodynamic therapy agent is selected from the group consisting of a porphyrin, a porphyrin isomer, and an expanded porphyrins.

32. The lipoprotein nanoplatform of claim 31, wherein said photodynamic therapy agent is selected from the group consisting of SiNc-BOA, SiPc-BOA, and pyropheophorbide-cholesterol ester (Pyro-CE).

33. The lipoprotein nanoplatform of claim 29, wherein said anticancer agent is a chemotherapeutic agent.

34. The lipoprotein nanoplatform of claim 28, wherein said anticancer agent is selected from the group consisting of alkylators, anthracyclines, antibiotics, aromatase inhibitors, bisphosphonates, cyclo-oxygnase inhibitors, estrogen receptor modulators, folate antagonists, inorganic arsenates, microtubule inhibitors, modifiers, nitrosureas, nucleoside analogs, osteoclast inhibitors, platinum containing compounds, retinoids, topoisomerase 1 inhibitors, tyrosine kinase inhibitors, and epidermal growth factor inhibitors.

35. The lipoprotein nanoplatform of claim 33, wherein said chemotherapeutic agent is selected from the group consisting of paclitaxel, cyclophosphoramide, docosahexaenoic acid (DHA)-paclitaxel conjugates, betulinic acid, and doxorubicin.

36. The lipoprotein nanoplatform of claim 27, wherein said therapeutic agent is an antiglaucoma drug, an anti-clotting agent, an anti-inflammatory drug, an anti-asthmatic, an antibiotic, an antifungal or an antiviral drug.

37. The lipoprotein nanoplatform of claim 36, wherein said therapeutic agent is an antiglaucoma drug.

38. The lipoprotein nanoplatform of claim 37, wherein said anti-glaucoma drug is selected from the group consisting of β-blockers such as timolol-base, betaxolol, atenolol, livobunolol, epinephrine, dipivalyl, oxonolol, acetazolamide-base and methzolamide.

39. The lipoprotein nanoplatform of claim 36, wherein said therapeutic agent is an anti-inflammatory drug.

40. The lipoprotein nanoplatform of claim 39, wherein said anti-inflammatory drug is a steroidal drug.

41. The lipoprotein nanoplatform of claim 40, wherein said steroidal drug is cortisone or dexamethasone

42. The lipoprotein nanoplatform of claim 36, wherein said anti-inflammatory drug is a non-steroidal drug.

43. The lipoprotein nanoplatform of claim 42, wherein said non-steroidal anti-inflammatory drugs (NSAID) is selected from the group consisting of piroxicam, indomethacin, naproxen, phenylbutazone, ibuprofen and diclofenac acid.

44. The lipoprotein nanoplatform of claim 36, wherein said therapeutic agent is an anti-asthmatic.

45. The lipoprotein nanoplatform of claim 36, wherein said anti-asthmatic is prednisolone or prednisone.

46. The lipoprotein nanoplatform of claim 36, wherein said antibiotic is chloramphenicol.

47. The lipoprotein nanoplatform of claim 46, wherein said antibiotic is selected from the group consisting of nystatin, amphotericin B, miconazole, Acyclovir™.

48. The lipoprotein nanoplatform of claim 1, wherein said apoprotein is selected from the group consisting of apoB-100, apoB-48, apoC, apoE and apoA.

49. The lipoprotein nanoplatform of claim 1, wherein said nanoplatform further comprises one or more triacylglycerols.

50. The lipoprotein nanoplatform of claim 1, wherein said nanoplatform further comprises a sterol, a sterol ester, or combinations thereof.

51. A pharmaceutical formulation comprising the non-naturally occurring lipoprotein based nanoplatform of claim 1.

52. A method of making the non-naturally occurring lipoprotein based nanoplatform of claim 1, comprising reconstituting a lipoprotein particle with an active agent and attaching a cell surface receptor ligand to the apoprotein of the reconstituted lipoprotein particle.

53. The method of claim 52, wherein said lipoprotein particle is an LDL particle.

54. The method of claim 52, wherein said lipoprotein particle is an HDL particle.

55. The method of claim 52, wherein said cell surface receptor ligand is a folate receptor ligand.
